(19) United States
(12) Patent Application Publication
(10) Pub. No.: US 2023/0313161 A1
(43) Pub. Date: Oct. 5, 2023

(54) DIET CONTROLLED EXPRESSION OF A NUCLEIC ACID ENCODING CAS9 NUCLEASE AND USES THEREOF

(71) Applicants: INSERM (INSTITUT NATIONAL DE LA SANTE ET DE LA RECHERCHE MEDICALE), PARIS (FR); ICM (INSTITUT DU CERVEAU ET DE LA MOELLE EPINIÈRE), PARIS (FR); CENTRE NATIONAL DE LE RECHERCHE SCIENTITIQUE (CNRS), PARIS (FR); SORBONNE UNIVERSITE, PARIS (FR); COMMISSARIAT À L'ÉNERGIE ATOMIQUE ET AUX ÉNERGIES AL TERNATIVES (CEA), PARIS (FR); ASSISTANCE PUBLIQUE - HÔPITAUX DE PARIS, PARIS (FR)

(72) Inventors: Philippe RAVASSARD, Paris (FR); Jacques MALLET, Paris (FR); Ché SERGUERA, Paris (FR)

(21) Appl. No.: 18/153,490

(22) Filed: Jan. 12, 2023

Related U.S. Application Data
(62) Division of application No. 16/304,988, filed on Nov. 27, 2018, now abandoned.

(30) Foreign Application Priority Data
Jun. 3, 2016 (EP) EP16172964

(51) Int. Cl. C12N 9/22; C12N 15/85; C12N 15/63; C12N 15/90

(52) U.S. Cl. CPC C12N 9/22; C12N 15/63; C12N 15/85; C12N 15/907; C12N 2830/002

(57) ABSTRACT

The present invention relates to a nucleic acid for the controlled expression of a nucleic acid encoding a Cas nuclease in an individual, comprising (i) a regulatory polynucleotide comprising a minimal promoter and from one to twenty AARE nucleic acids, and (ii) a nucleic acid encoding a Cas nuclease, which is placed under the control of the regulatory polynucleotide,

### SEQUENCE LISTING

This document incorporates by reference an electronic sequence listing file, which was electronically submitted along with this document. The XML file is named 2023-01-12_13390055US2_SeqListing, is 3,380 bytes, and was created on Jan. 11, 2023.

### FIELD OF THE INVENTION

The present invention relates to a nucleic acid for the controlled expression of a nucleic acid encoding Cas9 nuclease in an individual.

In particular, the expression of the nucleic acid may be controlled upon consumption of a diet deficient in at least one essential amino acid.

### BACKGROUND OF THE INVENTION

Genome editing using targetable nucleases is an emerging technology for the precise genome modification of organisms ranging from bacteria to plants and animals, including humans. Its attraction is that it can be used for almost all organisms in which targeted genome modification has not been possible with other kinds of methods.

Recent approaches to targeted genome modification, implementing e.g. zinc-finger nucleases (ZFNs), transcription-activator like effector nucleases (TALENs) and meganucleases, have enabled the scientific community to generate permanent mutations by introducing double-stranded breaks to activate repair pathways.

The capacity of designed nucleases, like ZFN and TALENs, to generate DNA double-stranded breaks at desired positions in the genome has created optimism for therapeutic translation of locus-directed genome engineering. However, these approaches are costly and time-consuming to engineer, limiting their widespread use, particularly for large scale, high-throughput studies.

More recently, a new tool based on a totally distinct and specific system, namely bacterial CRISPR-associated protein-9 nuclease (Cas9) from Streptococcus pyogenes has generated considerable interest.

Unlike the other gene-editing methods, it is cheap, quick and easy to use, and it has rapidly swept through laboratories around the world. The power of this system is to perform targeted, highly efficient alterations of genome sequence and gene expression that will undoubtedly transform all branches of biotechnology and spur the development of novel molecular therapeutics for human disease.

Following its initial demonstration in 2012, the CRISPR/Cas9 system has been widely adopted. This system has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671), zebrafish (Hwang et al., 2013, PLoS One, Vol. 8:e68708), C. elegans (Hai et al., 2014 Cell Res. doi: 10.1038/cr.2014.11), plants (Mali et al., 2013, Science, Vol. 339: 823-826), Xenopus tropicalis (Guo et al., 2014, Development, Vol. 141: 707-714), yeast (DiCarlo et al., 2013, Nucleic Acids Res., Vol. 41: 4336-4343), Drosophila (Gratz et al., 2014 Genetics, doi:10.1534/genetics.113.160713), monkeys (Niu et al., 2014, Cell, Vol. 156: 836-843), rabbits (Yang et al., 2014, J. Mol. Cell Biol., Vol. 6: 97-99), pigs (Hai et al., 2014, Cell Res. doi: 10.1038/cr.2014.11), rats (Ma et al., 2014, Cell Res., Vol. 24: 122-125) and mice (Mashiko et al., 2014, Dev. Growth Differ. Vol. 56: 122-129).

In addition, genome editing has been successfully applied to a number of diseases at the preclinical level, as well as in a phase I clinical trial (see the review by Cox et al., Nat Med. 2015 Feb;21(2):121-31). In evaluating the feasibility of a genome editing based therapy, the therapeutic effect of the desired genetic change should first be clearly established. Subsequently, the success of a given strategy will depend on the ease with which a therapeutic modification ‘threshold’ is achieved, a criteria that is governed by the fitness of edited cells, the DSB repair pathway utilized to edit the genome, and the efficiency of delivery of genome editing molecules to target cell types.

However, despite all its potential, CRISPR-Cas9 technology is presently seriously limited by the off-target effect associated with the editing process (i.e. genome editing in unwanted genomic localisation), and by the immunogenicity of the bacterial nuclease Cas9.

To date, the off-target issue appears to be inherent to the mechanistic features governing nuclease activities, as highlighted by Porteus (Genome Biology, 2015, 16:286). Porteus considers that an “important consideration in determining an appropriate delivery strategy is that genome editing, in contrast to gene-augmentation strategies, is a hit and run approach”. Furthermore, Porteus believes that “sustained expression of the nuclease not only is not needed but should be avoided: continued expression of a nuclease increases the probability of deleterious genomic instability and may either compromise the edited cell’s fitness or predispose the exposed cell to transformation”. Finally, Porteus concludes that “for therapeutic applications that require in vivo editing of cells, the challenge is greater and a solution has not been determined”.

A straightforward mean to alleviate the off-target that may be detrimental in some applications is to identify/design novel nucleases with greater specificities.

Therefore, there is a need in the art to provide new fine-tuned controlled expression systems for the expression of a nucleic acid encoding a Cas nuclease, in particular a Cas9 nuclease, in an individual, in particular for safe gene therapy approaches.

### SUMMARY OF THE INVENTION

One aspect of the invention relates to a nucleic acid for the controlled expression of a nucleic acid encoding a Cas nuclease in an individual, comprising:
a regulatory polynucleotide comprising a minimal promoter and at least one AARE (amino acid response element) nucleic acid, said regulatory polynucleotide being activated in an individual upon consumption of a diet deficient in at least one essential amino acid; and
a nucleic acid encoding a Cas nuclease, which is placed under the control of the said regulatory polynucleotide.

Another aspect of the invention relates to a nucleic acid vector for the controlled expression of a nucleic acid encoding a Cas nuclease, comprising a nucleic acid, as defined herein.

A still further aspect of the invention relates to a delivery particle comprising a nucleic acid or a nucleic acid vector, as defined herein.

In another aspect, the invention also relates to a pharmaceutical composition comprising (i) a nucleic acid according or a nucleic acid vector or a delivery particle, as defined herein, and (ii) a pharmaceutically acceptable vehicle.

In a further aspect, the invention relates to a host cell comprising the nucleic acid or a nucleic acid vector, as defined herein.

Another aspect of the invention relates to a pharmaceutical composition, as defined herein, for use as a medicament.

In a further aspect, the invention also relates to a pharmaceutical composition, as defined herein, for use as an active agent for editing the genome into at least one target cell.

In one aspect, the invention relates to a method for editing the genome into at least one target cell comprising at least the step of administering to an individual in need thereof the pharmaceutical composition, as defined herein.

In another aspect, the invention relates to a pharmaceutical composition, as defined herein, for use as an active agent for preventing and/or treating a disease.

An aspect of the invention also relates to a method for preventing and/or treating a disease comprising at least the step of administering to an individual in need thereof the pharmaceutical composition, as defined herein.

Finally, in a further aspect, the invention concerns a kit for treating and/or preventing a disease comprising:
a pharmaceutical composition, as defined herein, and
a pharmaceutically active compound.

### LEGENDS OF THE FIGURES

FIG. 1: Scheme illustrating the GCN2-eIF2α-ATF4 signalling pathway. In response to EAA starvation, activated GCN2 phosphorylates eIF2α, leading to an up-regulation of the transcription factor ATF4 and its recruitment to AARE sequences to induce target gene expression.

FIG. 2: Scheme illustrating the depiction of the AARE-Cas nuclease construct: six copies of the AAREs from Trb3 (black spots) promoter and the Tk minimal promoter compose this construct.

FIG. 3: Scheme illustrating the pTrip-2XAARE-NLS-FLAG-CAS9 plasmid. pTK indicates the position of the minimal TK promoter; 2X AARE indicates the position of the AARE nucleic acids; arrow “NLS-FLAG-CAS9” indicates the position of the nucleic acid encoding the Cas9 nuclease; arrow “AmpR” stands for the nucleic acid encoding for ampicillin resistance.

FIG. 4: Scheme illustrating the pTRIP blast_U6 AAVS1_2xAARE-Cas9-Flag-RFP plasmid. The lower panel is in continuity with the upper panel. The EcoR1 restriction site on the right end of the upper panel refers to the EcoR1 restriction site on the left end of the lower panel.

FIG. 5: Plots illustrating the Cas9 expression in 293T cells upon induction at T0 with either a medium deprived in Leucine (293T-C9 Leu-; plain curve) or a medium comprising tunicamycin (293T-C9 TU; dashed curve). Induction is performed at T0 and removed at 24 h. Expression is monitored 24 h and 48 h after removal of the induction, i.e. at T0+48 h and T0+72 h, respectively. The abscissa axis represents the time line (in hours) and the ordinate axis represents the band intensity for Cas9 nuclease, hence is representative of the Cas9 expression. The maximum expression of Cas9 is observed after 24 h upon induction, which arbitrarily represents 100% of expression.

FIG. 6: Plots illustrating the integration of a donor DNA (Do) at the AAVS1 site of the genome of 293T cells. 293T cells were transfected with the plasmid ‘pTRIP blast_U6 AAVS1-2xAARE-Cas9-flag-RFP’ (C9) as well as with the donor plasmid containing a cassette ‘AAVS1 cut site-GFP-p2a-Puromycin_AAVS1 cut site’ (Do). The number of puromycin resistant cells (ordinate axis) are counted upon induction in the presence of tunicamycin (293+Do+C9i Tu), or with a leucine-deprived medium (293+Do+C9i Leu-). As a control, 293T cells transfected with both plasmids (Do and C9) are assayed in the absence of induction (293+Do+C9 ni). Finally, 293T cells without any copy of the C9 plasmid were transfected with the Donor plasmid (Do) and the number of puromycin resistant cells was further counted.

FIG. 7: Plots illustrating the integration of a donor DNA (Do) at the AAVS 1 site of the genome of 293T cells containing one copy of the C9 plasmid (293-C9 cells), similarly as in FIG. 6. 293-C9 cells were transfected with the donor plasmid containing a cassette ‘AAVS1 cut site-GFP-p2a-Puromycin_AAVS1 cut site’ (Do). The number of puromycin resistant cells (ordinate axis) are counted upon induction in the presence of tunicamycin (293_C9+Doi Tu), or with a leucine-deprived medium (293_C9+Doi Leu-). As a control, 293-C9 cells transfected with plasmid Do are assayed in the absence of induction (293_C9+Do-ni). Finally, the number of puromycin resistant 293-C9 cells, transfected with the Donor plasmid (Do), in the absence of induction was further counted.

### DETAILED DESCRIPTION OF THE INVENTION

Any citation mentioned herein is incorporated by reference.

The inventors assessed the remarkable features of the nutritional adaptation pathway to a diet deprived of one essential amino acid to achieve a regulatory system ideally suited for gene therapy. The inventors found that such a system, based on dietary specific amino acid starvation, does not require the expression of synthetic transcription factors or regulatory proteins nor the administration of pharmacological inducers. It is physiological, non-toxic and is amenable to clinical application. This novel nutrition-based regulatory system stands as a physiological approach with the ability to resolve one of the major remaining hurdles in human gene therapy.

Without wishing to be bound to a theory, the inventors consider that the controlled expression system disclosed herein is particularly suitable system for a fine-tuned expression of a Cas nuclease (CRISPR (Clustered regularly interspaced short palindromic repeats) associated protein).

It is to be noted that WO 2013/068096 disclosed such a controlled expression system for several proteins, and the proof of concept was performed with the expression of the luciferase protein. Chaveroux et al. (Science Signaling, 2015, vol. 8(374), 1-10) took advantage of this system for characterizing the eIF2alpha-ATF4 signalling pathway.

However, due to the constraints of expressing Cas nuclease in a target host cell, e.g. absence of leakage, it could not be anticipated that the nutrition-based regulatory system disclosed in WO 2013/068096 and in Chaveroux et al. would provide a suitable tool for the controlled expression of a Cas nuclease.

The nucleic acid for the controlled expression of a nucleic acid encoding a Cas nuclease, as disclosed herein, allows for limiting or avoiding the off-targets, which are usually observed because of a lack of an efficiently controlled expression system (expression “leakage”).

### Nucleic Acid for the Controlled Expression of a Nucleic Acid Encoding Cas Nuclease

A first aspect of the invention concerns a nucleic acid for the controlled expression of a nucleic acid encoding a Cas nuclease in an individual, comprising:
a regulatory polynucleotide comprising a minimal promoter and at least one AARE (amino acid response element) nucleic acid, said regulatory polynucleotide being activated in an individual upon consumption of a diet deficient in at least one essential amino acid; and
a nucleic acid encoding a Cas nuclease, which is placed under the control of the said regulatory polynucleotide.

In another aspect, the invention also concerns a nucleic acid for the controlled expression of a nucleic acid encoding a Cas nuclease in at least one target cell of an individual, comprising:
a regulatory polynucleotide comprising a minimal promoter and at least one AARE (amino acid response element) nucleic acid, said regulatory polynucleotide being activated in an individual upon consumption of a diet deficient in at least one essential amino acid; and
a nucleic acid encoding a Cas nuclease, which is placed under the control of the said regulatory polynucleotide.

Within the scope of the instant invention, the expression “controlled expression” expression is intended to mean that the expression is induced or turned “on” and shut down or turned “off” in a precise manner, with respect to the moment of induction, the duration of induction.

In some embodiments, the Cas nuclease is selected in a group comprising a class I Cas nuclease, a class II Cas nuclease and a class III Cas nuclease.

For type I, type II or type III Cas proteins, the skilled artisan may refer to Chylinski et al. (2014, Nucleic Acids Research, Vol. 42(10) : 6091-6105); Sinkunas et al. (2011, The EMBO Journal, Vol. 30(7) : 1335-1342); Aliyari et al. (2009, Immunological Reviews, Vol. 227(1) : 176-188); Cass et al. (Biosci Rep, doi:10.1042/BSR20150043), Makarova et al. (2011, Biology Direct, Vol. 6 : 38); Gasiunas et al. (2012, Proc Natl Acad Sci USA, Vol. 109(39) : E2579-E2586) ; Heler et al. (2015, Nature, Vol. 519(7542) : 199-202); Esvelt et al. (2013, Nat Methods, Vol. 10(11) : doi :10.138/nmeth.2681), Zetsche et al. (Cell. 2015 Oct 22;163(3):759-71), or Chylinski et al. (2013, Biology, Vol. 10(5) : 726-737).

In some embodiments, a class I Cas nuclease is selected in a group comprising Cas3, Cas8a, Cas8b, Cas8c, Cas10d, Cse1 and Csy1.

In some embodiments, a class II Cas nuclease is selected in a group comprising Cas9, Cpf1, Csn2 and Cas4.

In some embodiments, a class III Cas nuclease is selected in a group comprising Cas10, Csm2 and Cmr5.

In some embodiments, the Cas nuclease is Cas9 nuclease.

In some embodiments, the Cas9 nuclease may originate from a bacterial source, in particular a bacterium selected in a group comprising Acaryochloris marina, Actinomyces naeslundii, Alcanivorax dieselolei, Belliella baltica, Campylobacter jejuni, Corynebacterium diphtheriae, Coriobacterium glomerans, Corynebacterium ulcerans, Desulfomonile tiedjei, Dickeya dadantii, Escherichia coli, Francisella tularensis, Lactobacillus kefiranofaciens, Listeria innocua, Methylobacterium extorquens, Micrococcus luteus, Myxococcus fulvus, Neisseria meningitidis, Pasteurella multocida, Prevotella intermedia, Prochlorococcus marinus, Psychroflexus torquis, Sphaerobacter thermophilus, Sphingobacterium sp., Staphylococcus aureus, Streptococcus mutans, Streptococcus pneumoniae, Streptococcus pyogenes, Streptococcus thermophilus and Streptomyces bingchenggensis.

In some embodiments, the Cas9 nuclease may originate from an archaebacterial source, such as e.g. Methanoculleus bourgensis.

Without any limitation, the Cas9 nuclease disclosed herein encompasses homologs, paralogs and orthologs and variants of naturally occurring Cas9 nucleases.

In certain embodiments, the Cas9 variants may include SpCas9-HF1 (Kleinstiver et al.; Nature. 2016 Jan 28;529(7587):490-5), fCas9, which is a fusion of catalytically inactive Cas9 to FokI nuclease (Guilinger et al.; Nat. Biotechnol. 2014: 32(6): 577-582), and any rationally engineered Cas9 nucleases with improved specificity as disclosed by Slaymaker et al. (Science. 2016 Jan 1;351(6268):84-8).

In some embodiments, the nucleic acid encoded a Cas9 nuclease and/or vectors encoding a Cas9 nuclease may be commercially available, e.g. from SIGMA-ALDRICH®.

In some other embodiments, Cas nucleases may be identified by the means of methods for the directed evolution of proteins Packer and Liu (Nat Rev Genet. 2015 Jul;16(7):379-94).

In some embodiments, the Cas nuclease is a DNA or RNA guided Cas nuclease.

Within the scope of the invention, “DNA or RNA guided” is intended to mean that in the presence of a guide DNA or RNA, the Cas nuclease is targeted to a nucleic acid, which sequence is complementary with the guide DNA and RNA. In certain embodiments, the expression of a nucleic acid encoding a Cas nuclease may be measured by any suitable method available in the state of the art, including the measure of the mRNA expression, resulting from the transcription of the nucleic acid encoding a Cas nuclease, and/or the measure of the Cas nuclease expression.

In some embodiments, the measure of the Cas nuclease expression may be performed by measuring the expression of the Cas nuclease with anti- antibodies that specifically bind to said Cas nuclease.

Within the scope of the present invention, an induced expression may be expressed as a time fold expression as compared to the basal, non-induced expression.

In some embodiments, the induced expression may vary from 2 fold to 10,000 fold, preferably from 4 fold to 500 fold, more preferably from 8 fold to 250 fold, most preferably from 10 fold to 100 fold, as compared to the basal expression.

Within the scope of the invention, from 2 fold to 10,000 fold includes 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 75 fold, 100 fold, 150 fold, 200 fold, 250 fold, 300 fold, 350 fold, 400 fold, 450 fold, 500 fold, 550 fold, 600 fold, 750 fold, 800 fold, 850 fold, 900 fold, 950 fold, 1,000 fold, 2,000 fold, 3,000 fold, 4,000 fold, 5,000 fold, 6,000 fold, 7,000 fold, 8,000 fold and 9,000 fold.

Within the scope of the invention, the expression “minimal promoter” is intended to mean a promoter including all the required elements to properly initiate transcription of a gene of interest positioned downstream. Within the scope of the invention, “minimal promoter” and “core promoter” are considered as equivalent expressions. A skilled artisan understands that the “minimal promoter” includes at least a transcription start site, a binding site for a RNA polymerase and a binding site for general transcription factors (TATA box).

Suitable minimal promoters are known for a skilled artisan.

In some embodiments, a minimal promoter suitable for carrying out the invention may be selected in a group comprising the promoter of the thymidine kinase, the promoter of the β-globin, the promoter for cytomegalovirus (CMV), the SV40 promoter and the like.

In some embodiments, the individual is a human or a non-human mammal, preferably a human.

In some embodiments, the non-human mammal is selected in a group comprising a pet such as a dog, a cat, a domesticated pig, a rabbit, a ferret, a hamster, a mouse, a rat and the like; a primate such as a chimp, a monkey, and the like; an economically important animal such as cattle, a pig, a rabbit, a horse, a sheep, a goat, a mouse, a rat.

Within the scope of the invention a “target cell” is intended to refer to a cell from the said individual, for which an expression of a Cas nuclease would be beneficial.

Within the scope of the present invention, the expression “essential amino acid” includes histidine (His, H), isoleucine (Ile, I), leucine (Leu, L), Lysine (Lys, K), methionine (Met, M), phenylalanine (Phe, F), threonine (Thr, T), tryptophane (Trp, W) and valine (Val, V).

Within the scope of the invention, the expression “at least one essential amino acid” is intended to mean 1, 2, 3, 4, 5, 6, 7, 8 or 9 essential amino acid(s).

In some embodiments, a diet deficient in at least one essential amino acid may be administered to an individual for a time period of 5 min to 12 h, which includes 10 min, 15 min, 20 min, 25 min, 30 min, 45 min, 1 h, 1 h 30 min, 2 h, 2 h 30 min, 3 h, 3 h 30 min, 4 h, 4 h 30 min, 5 h, 5 h 30 min, 6 h, 6 h 30 min, 7 h, 7 h 30 min, 8 h, 8 h 30 min, 9 h, 9 h 30 min, 10 h, 10 h 30 min, 11 h, 11 h 30 min.

In some embodiments, a diet deficient in at least one essential amino acid may be administered to an individual once, twice, three times, four times, five times, six times a day, or more.

In certain embodiments, the diet deficient in at least one essential amino acid may be administered to an individual once or twice a day.

In some embodiments, the diet deficient in at least one essential amino acid may be administered to an individual early in the morning, e.g. for breakfast, and then the individual may be administered a normal diet for lunch and dinner.

Within the scope of the instant invention, the expression “normal diet” is intended to mean a diet that is not deficient in any of the essential amino acids.

In some embodiments, a diet deficient in at least one essential amino acid may be administered to an individual every day, every other day, once a week, twice a week, three times per week.

In some embodiments, a diet deficient in at least one essential amino acid may be administered to an individual for a period of half a day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days 20 days, or more.

In some embodiments, a diet deficient in at least one essential amino acid may be repeated every week, every other week, every month, every month, or more.

In some embodiment, the diet deficient in at least one essential amino acid may be provided by an isoleucine-free, leucine-free and valine-free powdered food product commercially available from NUTRICA METABOLICS®, under the name MILUPA®. This diet is adapted to individual having Maple syrup urine disease, which disease appears to affect the branched chain amino acid metabolism.

In certain embodiment, a leucine-free, isoleucine-free or valine-free diet may be obtained by mixing the isoleucine-free, leucine-free and valine-free powder with an external source for the 2 remaining amino acids.

In certain embodiments, a phenylalanine-free diet may be provided a phenylalanine-free powder, commercially available from MEAD JOHNSON®. This diet is adapted to individual having phenylketonuria.

In practice, the powder is mixed with an adapted a liquid or a semi-solid food that is free of the desired essential amino acid.

In certain embodiment, an amino acids starvation can be mimicked by the administration of Halofuginone, or under any other name corresponding to the molecule “4(3H)-Quinazolinone, 7-bromo-6-chloro-3-[3-(3-hydroxy-2-piperidinyl)-2-oxopropyl]-, trans-(±)-, or commercialized as for example, Halocur, Stenorol, Flavomycin, Lincomix, Stafac.

In one embodiment, the amino acid response element (AARE) nucleic acid is selected in a group comprising a nucleic acid of sequence SEQ ID NO: 1, SEQ ID NO: 2, SEQ ID NO: 3, SEQ ID NO: 4 and SEQ ID NO: 5.

Within the scope of the instant invention the expression “at least one AARE nucleic acid” includes at least 2, at least 3, at least 4 and at least 5 AARE nucleic acids. The expression “at least one AARE nucleic acid” thus includes 1, 2, 3, 4, 5, 6, 7, 8, 9, 10, 11, 12, 13, 14, 15, 16, 17, 18, 19 and 20 AARE nucleic acids.

In certain embodiments, the regulatory polynucleotide comprises at least two AARE nucleic acids.

In some other embodiments, the regulatory polynucleotide comprises from one to twenty AARE nucleic acids, preferably from two to ten AARE nucleic acids.

In certain embodiments, the regulatory polynucleotide comprises from two to six AARE nucleic acids.

In some embodiments, the regulatory polynucleotide comprises two AARE nucleic acids selected in the group comprising a nucleic acid of sequence SEQ ID NO: 2 and SEQ ID NO: 4.

In some embodiments, the regulatory polynucleotide comprises six AARE nucleic acids of sequence SEQ ID NO: 1.

In certain embodiments, the two AARE nucleic acids, or alternatively, the at least two AARE nucleic acids may be identical or distinct.

In some embodiments, the regulatory polynucleotide comprised in the nucleic acid for the controlled expression of a nucleic acid encoding a Cas nuclease may also be activated upon administration to an individual of halofuginone, tunicamycin, and the like, i.e. compounds which are known to have activating properties of the AARE nucleic acids.

### Nucleic Acid Vector

In another aspect, the invention also concerns a nucleic acid vector for the controlled expression of a nucleic acid encoding a Cas nuclease, comprising a nucleic acid for the controlled expression of a nucleic acid encoding a Cas nuclease, as defined herein.

In some embodiments, the nucleic acid for the controlled expression of a nucleic acid encoding a Cas nuclease according to the invention is incorporated in a vector that is suitable for gene therapy.

Within the scope of the instant invention, the expression “vector that is suitable for gene therapy” is intended to mean that the vector comprises the essential elements for achieving the expression of the nucleic acid encoding a Cas nuclease in a target cell.

In certain embodiments, the vector is a viral vector.

In some embodiments, a viral vector is selected in a group comprising an adenoviruse, an adeno-associated virus (AAV), an alphavirus, a herpesvirus, a lentivirus, a non-integrative lentivirus, a retrovirus, vaccinia virus and a bacculovirus.

### Delivery Particle

In some embodiments, the nucleic acid for the controlled expression of a nucleic acid encoding a Cas nuclease or the nucleic acid vector, as defined herein, may be comprised in a particle, in particular, in association other compounds, such as e.g. with lipids, protein, peptides, or polymers.

Within the scope of the invention said particle, or “delivery particle” is intended to provide, or “deliver”, the target cells with the nucleic acid encoding a Cas nuclease or the nucleic acid vector comprising the said nucleic acid encoding a Cas nuclease.

In a still other aspect, the invention further concerns a delivery particle comprising a nucleic acid for the controlled expression of a nucleic acid encoding a Cas nuclease or a nucleic acid vector, as defined herein.

In certain embodiments, the delivery particle may be in the form of a lipoplexe, comprising cationic lipids; a lipid nano-emulsion; a solid lipid nanoparticle; a peptide based particle; a polymer based particle, in particular comprising natural and/or synthetic polymers.

In some embodiments, a polymer based particle may comprise a protein; a peptide; a polysaccharide, in particular chitosan.

In some embodiments, a polymer based particle may comprise a synthetic polymer, in particular, a polyethylene imine (PEI), a dendrimer, a poly (DL- Lactide) (PLA), a poly(DL-Lactide-co-glycoside) (PLGA), a polymethacrylate and a polyphosphoesters.

In some embodiments, the delivery particle further comprises at its surface one or more ligands suitable for binding to a target receptor exposed at the membrane of a targeted cell.

### Pharmaceutical Composition

Another aspect of the present invention concerns a pharmaceutical composition comprising (i) a nucleic acid for the controlled expression of a nucleic acid encoding a Cas nuclease, or a nucleic acid vector or a delivery particle, as defined herein, and (ii) a pharmaceutically acceptable vehicle.

The formulation of pharmaceutical compositions according to the instant invention is well known to persons skilled in the art.

As referred herein, a nucleic acid for the controlled expression of a nucleic acid encoding a Cas nuclease, or a nucleic acid vector or a delivery particle, as defined in the present disclosure, may represent the active agent.

In some embodiments, the pharmaceutical composition may comprise a nucleic acid for the controlled expression of a nucleic acid encoding a Cas nuclease, or a nucleic acid vector or a delivery particle, as defined in the present disclosure, as the only active agent.

In some embodiments, a suitable pharmaceutically acceptable vehicle according to the invention includes any and all conventional solvents, dispersion media, fillers, solid carriers, aqueous solutions, coatings, antibacterial and antifungal agents, isotonic and absorption delaying agents, and the like.

In certain embodiments, suitable pharmaceutically acceptable vehicles may include, water, saline, phosphate buffered saline, dextrose, glycerol, ethanol and a mixture thereof.

In some embodiments, pharmaceutically acceptable vehicles may further comprise minor amounts of auxiliary substances such as wetting or emulsifying agents, preservatives or buffers, which enhance the shelf life or effectiveness of the cells. The preparation and use of pharmaceutically acceptable vehicles is well known in the art.

Except insofar as any conventional media or agent is incompatible with the active ingredient, use thereof in the pharmaceutical compositions of the present invention is contemplated.

In some embodiments, the pharmaceutical composition may be administered to an individual in need thereof by any route, i.e. by an oral administration, a topical administration or a parenteral administration, e.g., by injection, including a sub-cutaneous administration, a venous administration, an arterial administration, in intra-muscular administration, an intra-ocular administration and an intra-auricular administration.

In certain embodiments, the administration of the pharmaceutical composition by injection may be directly performed in the target tissue of interest, in particular in order to avoid spreading of the nucleic acid or the nucleic acid vector comprised in the said pharmaceutical composition.

The inventors consider that this is particularly important when the brain tissue is target. Nucleic acid vector infusions can be conducted with great precision in specific parts of the brain tissue, e.g. by the mean of taking advantage of a magnetic resonance scanner, in particular using frameless stereotactic aiming devices. The use of MRI-guidance and new stereotactic aiming devices, have now established a strong foundation for neurological gene therapy to become an accepted procedure in interventional neurology.

Other modes of administration employ pulmonary formulations, suppositories, and transdermal applications.

In some embodiments, an oral formulation according to the invention includes usual excipients, such as, for example, pharmaceutical grades of mannitol, lactose, starch, magnesium stearate, sodium saccharine, cellulose, magnesium carbonate, and the like.

In some embodiments, an effective amount of said compound is administered to said individual in need thereof.

Within the scope of the instant invention, an “effective amount” refers to the amount of said compound that alone stimulates the desired outcome, i.e. alleviates or eradicates the symptoms of the encompassed disease, in particular a genetic disorder.

It is within the common knowledge of a skilled artisan to determine the effective amount of a nucleic acid for the controlled expression of a nucleic acid encoding a Cas nuclease, or a nucleic acid vector or a delivery particle in order to observe the desired outcome.

Within the scope of the instant invention, the effective amount of the compound to be administered may be determined by a physician or an authorized person skilled in the art and can be suitably adapted within the time course of the treatment.

In certain embodiments, the effective amount to be administered may depend upon a variety of parameters, including the material selected for administration, whether the administration is in single or multiple doses, and the individual’s parameters including age, physical conditions, size, weight, gender, and the severity of the disease to be treated.

In certain embodiments, an effective amount of the active agent may comprise from about 0.001 mg to about 3000 mg, per dosage unit, preferably from about 0.05 mg to about 100 mg, per dosage unit.

Within the scope of the instant invention, from about 0.001 mg to about 3000 mg includes, from about 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg and 2950 mg, per dosage unit.

In certain embodiments, the active agent may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In some particular embodiments, an effective amount of the active agent may comprise from about 1×10⁵ to about 1×10¹⁵ copies of the nucleic acid for the controlled expression of a nucleic acid encoding a Cas nuclease, or the nucleic acid vector or the delivery particle, as defined in the present disclosure, per dosage unit.

Within the scope of the instant invention, from about 1×10⁵ to about 1×10¹⁵ copies includes 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ copies, per dosage unit.

### Target Cell and Host Cell

In a further aspect, the invention concerns a host cell comprising the nucleic acid for the controlled expression of a nucleic acid encoding a Cas nuclease or a nucleic acid vector, as defined herein.

The target cell and/or the host cell may be selected among a prokaryotic cell or an eukaryotic cell.

Within the scope of the invention, a “prokaryotic cell” encompasses a bacterial cell and an archaebacterial cell.

In some embodiments, the target cell and/or the host cell is a eukaryotic cell.

Within the scope of the invention, a “eukaryotic cell” encompasses a yeast, an algae cell, a plant cell, an animal cell, preferably a mammal cell and more preferably a human cell.

In some preferred embodiments, the eukaryotic cell is a mammal cell, preferably a human cell.

In certain embodiments, a target cell and/or a host cell according to the instant invention may encompass, without limitation, a cell of the central nervous system, an epithelial cell, a muscular cell, an embryonic cell, a germ cell, a stem cell, a progenitor cell, a hematopoietic stem cell, a hematopoietic progenitor cell, an induced Pluripotent Stem Cell (iPSC).

In some particular embodiments, the target cell and/or the host cell is not a stem cell, a progenitor cell, a germinal cell or an embryonic cell.

In some embodiments, the target cell and/or the host cell may belong to a tissue selected in a group comprising a muscle tissue, a nervous tissue, a connective tissue, and an epithelial tissue.

In some embodiments, the target cell and/or the host cell may belong to an organ selected in a group comprising a bladder, a bone, a brain, a breast, a central nervous system, a cervix, a colon, an endometrium, a kidney, a larynx, a liver, a lung, an oesophagus, an ovarian, a pancreas, a pleura, a prostate, a rectum, a retina, a salivary gland, a skin, a small intestine, a soft tissue, a stomach, a testis, a thyroid, an uterus, a vagina.

### Uses

Another aspect of the invention concerns a pharmaceutical composition comprising a nucleic acid for the controlled expression of a nucleic acid encoding a Cas nuclease, or a nucleic acid vector, or a delivery particle, as defined herein, and a pharmaceutically acceptable vehicle, for use as a medicament.

In one aspect, the invention also relates to the use of a nucleic acid for the controlled expression of a nucleic acid encoding a Cas nuclease, as defined herein, for the preparation or the manufacture of a medicament.

In a still other aspect, the invention concerns a pharmaceutical composition comprising a nucleic acid for the controlled expression of a nucleic acid encoding a Cas nuclease, or a nucleic acid vector, or a delivery particle, as defined herein, and a pharmaceutically acceptable vehicle, for use as an active agent for editing the genome into at least one target cell.

Another aspect of the invention further relates to the use of a nucleic acid for the controlled expression of a nucleic acid encoding a Cas nuclease, as defined herein, as an active agent for editing the genome into at least one target cell.

In certain embodiments, the edition of the genome may be performed in vivo, in vitro or ex vivo.

In some embodiments, the edition of the genome may be performed as in Komor et al. Nature; 2016 Apr 20;533(7603):420-4.

In one embodiment, the target cell has at least a genetic mutation.

In some embodiments, the genetic mutation is present in a gene selected in a group comprising MTTP; CNGB3; SLC39A4; TRMU; ACOX1; ADA; ABCD1; SAMHD1; MAN2B1; HBA; ATRX; COL4A3; COL4A4; COL4A5; ALMS1; SLC12A6; ASL; CYP19A1; SLC35A3; ASNS; AGA; TTPA; ATM; SACS; BBS10; BBS1; BBS2, BBS12; CIITA; BSND; GP1BA; HSD3B2; ACAT1; GPR56; BTD; BLM; ASPA; CPS1; CPT1A; CPT2; RAB23; RMRP; SLC6A8; GAMT; CYP27A1; NDRG1; PRPS1; GJB1; VPS13A; CHM; CYBA; CYBB; SLC25A13; ASS1; VPS13B; ACSF3; GFM1; TSFM; PROP1; LHX3; PSAP; CYP17A1; MPL; PMM2; MPI; ALG6; NTRK1; CHRNE; RAPSN; HAX1; VPS45; SLC4A11; CYP11B2; CFTR; CTNS; HSD17B4; LOXHD1; DMD; RTEL1; COL7A1; ADAMTS2; EVC; EMD; NR2E3; ETHE1; GLA; F9; F11; IKBKAP; LDLR; LDLRAP1; ABCC8; KCNJ11; MEFV; FANCA; FANCC; FANCG; FMR1; FH; GALK1; GALT; GBA; SLC12A3; GCDH; ETFA; ETFDH; AMT; GLDC; G6PC; SLC37A4; GAA; AGL; GBE1; PYGM; PFKM; BCS1L; HFE2; TFR2; ALDOB; TECPR2; HPS1; HPS3; HMGCL; HLCS; CBS; MTHFR; MTRR; HYLS1; SLC25A15; EDA; ALPL; GNE; MED17; IVD; TMEM216; RGPRIP1L; LAMA3; LAMB3; LAMC2; GALC; TGM1; CEP290; RDH12; RPE65; LCA5; CRB1; LRPPRC; GLE1; EIF2B5; CAPN3; DYSF; SGCG; SGCA; SGCB; FKRP; DLD; STAR; LPL; HADHA; SLC7A7; BCKDHA; BCKDHB; MKS1; ACADM; MLC1; ATP7A; ARSA; MCCC1; MCCC2; OPA3; MMAA; MMAB; MUT; MMACHC; VSX2; ACAD9; NDUFAF5; NDUFS6; MPV17; PUS1; GNPTAG; MCOLN1; IDUA; IDS; NAGLU; HGSNAT; GNS; GLB1; HYAL1; ARSB; SUMF1; POMGNT1; TYMP; MTM1; NAGS; NEB; AQP2; NPHS1; NPHS2; CLN3; CLN5; CLN6; CLN8; MFSD8; PPT1; TPP1; SMPD1; NPC1; NPC2; NBN; GJB2; WNT10A; RAG2; DCLRE1C; OAT; OTC; TCIRG1; SLC26A4; PAH; PHGDH; PKHD1; AIRE; VRK1; RARS2; SLC22A5; DNAI1; DNAH5; DNAI2; AGXT; GRHPR; HOGA1; SEPSECS; ABCB11; PCCA; PCCB; CTSK; PDHA1; PDHB; PTS; ATP6V1B1; EYS; CERKL; FAM161A; DHDDS; PEX7; AGPS; ESCO2; SLC17A5; HEXB; SMARCAL1; TH; ALDH3A2; DHCR7; SMN1; MESP2; COL27A1; LIFR; SLC26A2; HEXA; FAH; MYO7A; USH1C; CDH23; PCDH15; USH2A; CLRN1; ACADVL; FKTN; ATP7B; LIPA; RS1; IL2RG; PEX1; PEX2; PEX6 and PEX10.

In one aspect, the present invention concerns a pharmaceutical composition comprising a nucleic acid for the controlled expression of a nucleic acid encoding a Cas nuclease, or a nucleic acid vector, or a delivery particle, as defined herein, and a pharmaceutically acceptable vehicle, for use as an active agent for treating and/or preventing a disease.

In some embodiments, the disease is selected in a group comprising a genetic disorder, an infectious disease and a cancer.

In some embodiments, the disease is a genetic disorder.

In certain embodiments, the genetic disorder is selected in a non-limiting group comprising Abetalipoproteinemia; Achromatopsia; Acrodermatitis Enteropathica; Acute Infantile Liver Failure; Acyl-CoA Oxidase I Deficiency; Adenosine Deaminase Deficiency; Adrenoleukodystrophy, X-Linked; Aicardi-Goutières Syndrome; Alpha-Mannosidosis; Alpha-Thalassemia; Alpha-Thalassemia Mental Retardation Syndrome; Alport Syndrome; Alstrom Syndrome; Andermann Syndrome; Argininosuccinic Aciduria; Aromatase Deficiency; Arthrogryposis, Mental Retardation, and Seizures; Asparagine Synthetase Deficiency; Aspartylglycosaminuria; Ataxia With Isolated Vitamin E Deficiency; Ataxia-Telangiectasia; Autosomal Recessive Spastic Ataxia of Charlevoix-Saguenay; Bardet-Biedl Syndrome; Bardet-Biedl Syndrome; Bare Lymphocyte Syndrome, Type II; Bartter Syndrome, Type 4A; Bernard-Soulier Syndrome, Type A1; Beta-Globin-Related Hemoglobinopathy; 3-Beta-Hydroxysteroid Dehydrogenase Type II Deficiency; Beta-Ketothiolase Deficiency; Bilateral Frontoparietal Polymicrogyria; Biotinidase Deficiency; Bloom Syndrome; Canavan Disease; Carbamoylphosphate Synthetase I Deficiency; Carnitine Palmitoyltransferase IA Deficiency; Carnitine Palmitoyltransferase II Deficiency; Carpenter Syndrome; Cartilage-Hair Hypoplasia; Cerebral Creatine Deficiency Syndrome 1; Cerebral Creatine Deficiency Syndrome 2; Cerebrotendinous Xanthomatosis; Charcot-Marie-Tooth Disease, Type 4D; Charcot-Marie-Tooth Disease, Type 5 / Arts syndrome; Charcot-Marie-Tooth Disease, X-Linked; Choreoacanthocytosis; Choroideremia; Chronic Granulomatous Disease; Chronic Granulomatous Disease; Citrin Deficiency; Citrullinemia, Type 1; Cohen Syndrome; Combined Malonic and Methylmalonic Aciduria; Combined Oxidative Phosphorylation Deficiency 1; Combined Oxidative Phosphorylation Deficiency 3; Combined Pituitary Hormone Deficiency 2; Combined Pituitary Hormone Deficiency 3; Combined SAP Deficiency; Congenital Adrenal Hyperplasia due to 17-Alpha-Hydroxylase Deficiency; Congenital Amegakaryocytic Thrombocytopenia; Congenital Disorder of Glycosylation, Type Ia; Congenital Disorder of Glycosylation, Type Ib; Congenital Disorder of Glycosylation, Type Ic; Congenital Insensitivity to Pain with Anhidrosis; Congenital Myasthenic Syndrome; Congenital Myasthenic Syndrome; Congenital Neutropenia; Congenital Neutropenia; Corneal Dystrophy and Perceptive Deafness; Corticosterone Methyloxidase Deficiency; Cystic Fibrosis; Cystinosis; D-Bifunctional Protein Deficiency; Deafness, Autosomal Recessive 77; Duchenne Muscular Dystrophy / Becker Muscular Dystrophy; Dyskeratosis Congenita; Dystrophic Epidermolysis Bullosa; Ehlers-Danlos Syndrome, Type VIIC; Ellis-van Creveld Syndrome; Emery-Dreifuss Myopathy 1; Enhanced S-Cone Syndrome; Ethylmalonic Encephalopathy; Fabry Disease; Factor IX Deficiency; Factor XI Deficiency; Familial Dysautonomia; Familial Hypercholesterolemia; Familial Hypercholesterolemia, Autosomal Recessive; Familial Hyperinsulinism; Familial Mediterranean Fever; Fanconi Anemia, Group A; Fanconi Anemia, Group C; Fanconi Anemia, Group G; Fragile X Syndrome; Fumarase Deficiency; Galactokinase Deficiency; Galactosemia; Gaucher Disease; Gitelman Syndrome; Glutaric Acidemia, Type I; Glutaric Acidemia, Type IIa; Glutaric Acidemia, Type IIc; Glycine Encephalopathy; Glycine Encephalopathy; Glycogen Storage Disease, Type Ia; Glycogen Storage Disease, Type Ib; Glycogen Storage Disease, Type II; Glycogen Storage Disease, Type III; Glycogen Storage Disease, Type IV / Adult Polyglucosan Body Disease; Glycogen Storage Disease, Type V; Glycogen Storage Disease, Type VII; GRACILE Syndrome and Other BCS1L-Related Disorders; Hemochromatosis, Type 2A; Hemochromatosis, Type 3; Hereditary Fructose Intolerance; Hereditary Spastic Paraparesis 49; Hermansky-Pudlak Syndrome, Type 1; Hermansky-Pudlak Syndrome, Type 3; HMG-CoA Lyase Deficiency; Holocarboxylase Synthetase Deficiency; Homocystinuria; Homocystinuria due to MTHFR Deficiency; Homocystinuria, cblE Type; Hydrolethalus Syndrome; Hyperornithinemia-Hyperammonemia-Homocitrullinuria Syndrome; Hypohidrotic Ectodermal Dysplasia 1; Hypophosphatasia; Inclusion Body Myopathy 2; Infantile Cerebral and Cerebellar Atrophy; Isovaleric Acidemia; Joubert Syndrome 2; Joubert Syndrome 7 / Meckel Syndrome 5 / COACH Syndrome; Junctional Epidermolysis Bullosa; Junctional Epidermolysis Bullosa; Junctional Epidermolysis Bullosa; Krabbe Disease; Lamellar Ichthyosis, Type 1; Leber Congenital Amaurosis 10 and Other CEP290-Related Ciliopathies; Leber Congenital Amaurosis 13; Leber Congenital Amaurosis 2 / Retinitis Pigmentosa 20; Leber Congenital Amaurosis 5; Leber Congenital Amaurosis 8 / Retinitis Pigmentosa 12 / Pigmented Paravenous Chorioretinal Atrophy; Leigh Syndrome, French-Canadian Type; Lethal Congenital Contracture Syndrome 1 / Lethal Arthrogryposis with Anterior Horn Cell Disease; Leukoencephalopathy with Vanishing White Matter; Limb-Girdle Muscular Dystrophy, Type 2A; Limb-Girdle Muscular Dystrophy, Type 2B; Limb-Girdle Muscular Dystrophy, Type 2C; Limb-Girdle Muscular Dystrophy, Type 2D; Limb-Girdle Muscular Dystrophy, Type 2E; Limb-Girdle Muscular Dystrophy, Type 2I; Lipoamide Dehydrogenase Deficiency; Lipoid Adrenal Hyperplasia; Lipoprotein Lipase Deficiency; Long-Chain 3-Hydroxyacyl-CoA Dehydrogenase Deficiency; Lysinuric Protein Intolerance; Maple Syrup Urine Disease, Type 1a; Maple Syrup Urine Disease, Type 1b; Meckel Syndrome 1 / Bardet-Biedl Syndrome 13; Medium Chain Acyl-CoA Dehydrogenase Deficiency; Megalencephalic Leukoencephalopathy with Subcortical Cysts; Menkes Disease; Metachromatic Leukodystrophy; 3-Methylcrotonyl-CoA Carboxylase Deficiency; 3-Methylcrotonyl-CoA Carboxylase Deficiency; 3-Methylglutaconic Aciduria, Type III / Optic Atrophy 3, with Cataract; Methylmalonic Acidemia; Methylmalonic Acidemia; Methylmalonic Acidemia; Methylmalonic Aciduria and Homocystinuria, Cobalamin C Type; Microphthalmia / Anophthalmia; Mitochondrial Complex I Deficiency; Mitochondrial Complex I Deficiency; Mitochondrial Complex I Deficiency; Mitochondrial DNA Depletion Syndrome 6 / Navajo Neurohepatopathy; Mitochondrial Myopathy and Sideroblastic Anemia 1; Mucolipidosis II / IIIA; Mucolipidosis III Gamma; Mucolipidosis IV; Mucopolysaccharidosis, Type I; Mucopolysaccharidosis, Type II; Mucopolysaccharidosis, Type IIIB; Mucopolysaccharidosis, Type IIIC; Mucopolysaccharidosis, Type IIID; Mucopolysaccharidosis, Type IVb / GM1 Gangliosidosis; Mucopolysaccharidosis, Type IX; Mucopolysaccharidosis, Type VI; Multiple Sulfatase Deficiency; Muscle-Eye-Brain Disease and Other POMGNT1-Related Congenital Muscular Dystrophy-Dystroglycanopathies; Myoneurogastrointestinal Encephalopathy; Myotubular Myopathy 1; N-Acetylglutamate Synthase Deficiency; Nemaline Myopathy 2; Nephrogenic Diabetes Insipidus, Type II; Nephrotic Syndrome / Congenital Finnish Nephrosis; Nephrotic Syndrome / Steroid-Resistant Nephrotic Syndrome; Neuronal Ceroid-Lipofuscinosis; Neuronal Ceroid-Lipofuscinosis; Niemann-Pick Disease, Type A/B; Niemann-Pick Disease, Type C; Nijmegen Breakage Syndrome; Non-Syndromic Hearing Loss; Odonto-Onycho-Dermal Dysplasia / Schopf-Schulz-Passarge Syndrome; Omenn Syndrome; Omenn Syndrome / Severe Combined Immunodeficiency, Athabaskan-Type; Ornithine Aminotransferase Deficiency; Ornithine Transcarbomylase Deficiency; Osteopetrosis 1; Pendred Syndrome; Phenylalanine Hydroxylase Deficiency; 3-Phosphoglycerate Dehydrogenase Deficiency; Polycystic Kidney Disease, Autosomal Recessive; Polyglandular Autoimmune Syndrome, Type 1; Pontocerebellar Hypoplasia, Type 1A; Pontocerebellar Hypoplasia, Type 6; Primary Carnitine Deficiency; Primary Ciliary Dyskinesia; Primary Hyperoxaluria, Type 1; Primary Hyperoxaluria, Type 2; Primary Hyperoxaluria, Type 3; Progressive Cerebello-Cerebral Atrophy; Progressive Familial Intrahepatic Cholestasis, Type 2; Propionic Acidemia; Propionic Acidemia; Pycnodysostosis; Pyruvate Dehydrogenase E1-Alpha Deficiency; Pyruvate Dehydrogenase E1-Beta Deficiency; 6-Pyruvoyl-Tetrahydropterin Synthase Deficiency; Renal Tubular Acidosis and Deafness; Retinitis Pigmentosa 25; Retinitis Pigmentosa 26; Retinitis Pigmentosa 28; Retinitis Pigmentosa 59; Rhizomelic Chondrodysplasia Punctata, Type 1; Rhizomelic Chondrodysplasia Punctata, Type 3; Roberts Syndrome; Salla Disease; Sandhoff Disease; Schimke Immunoosseous Dysplasia; Segawa Syndrome; Sjogren-Larsson Syndrome; Smith-Lemli-Opitz Syndrome; Spinal Muscular Atrophy; Spondylothoracic Dysostosis; Steel Syndrome; Stuve-Wiedemann Syndrome; Sulfate Transporter-Related Osteochondrodysplasia; Tay-Sachs Disease; Tyrosinemia, Type I; Usher Syndrome, Type IB; Usher Syndrome, Type IC; Usher Syndrome, Type ID; Usher Syndrome, Type IF; Usher Syndrome, Type IIA; Usher Syndrome, Type III; Very Long Chain Acyl-CoA Dehydrogenase Deficiency; Walker-Warburg Syndrome and Other FKTN-Related Dystrophies; Wilson Disease; Wolman Disease / Cholesteryl Ester Storage Disease; X-Linked Juvenile Retinoschisis; X-Linked Severe Combined Immunodeficiency and Zellweger Syndrome Spectrum.

In some embodiments, the disease is an infectious disease.

In certain embodiments, the infectious disease selected in a non-limiting group comprising Anaplasmosis; Anthrax; Babesiosis; Botulism; Brucellosis; Burkholderia mallei infection (glanders); Burkholderia pseudomallei infection (melioidosis); Campylobacteriosis; Carbapenem-resistant Enterobacteriaceae infection (CRE); Chancroid; Chikungunya infection; Chlamydia infection; Ciguatera; Clostridium difficile infection; Clostridium perfringens infection (Epsilon Toxin); Coccidioidomycosis fungal infection (Valley fever); Creutzfeldt-Jacob Disease, transmissible spongioform (CJD); Cryptosporidiosis; Cyclosporiasis; Dengue Fever; Diphtheria; E. Coli infection; Eastern Equine Encephalitis (EEE); Ebola Hemorrhagic Fever (Ebola); Ehrlichiosis; Arboviral or parainfectious encephalitis; Non-polio enterovirus infection; D68 enterovirus infection, (EV-D68); Giardiasis; Gonococcal infection (Gonorrhea); Granuloma inguinale; Type B Haemophilus Influenza disease, (Hib or H-flu); Hantavirus pulmonary syndrome (HPS); Hemolytic uremic syndrome (HUS); Hepatitis A (Hep A); Hepatitis B (Hep B); Hepatitis C (Hep C); Hepatitis D (Hep D); Hepatitis E (Hep E); Herpes; Herpes zoster, zoster VZV (Shingles); Histoplasmosis; Human Immunodeficiency Virus/AIDS (HIV/AIDS); Human Papillomarivus (HPV); Influenza (Flu); Lead poisoning; Legionellosis (Legionnaires Disease); Leprosy (Hansens Disease); Leptospirosis; Listeriosis; Lyme Disease; Lymphogranuloma venereum infection (LVG); Malaria; Measles; Viral meningitis; Meningococcal disease; Middle East respiratory syndrome coronavirus (MERS-CoV); Mumps; Norovirus; Paralytic shellfish poisoning; Pediculosis (lice, head and body lice); Pelvic inflammatory disease (PID); Pertussis; Bubonic, septicemic or pneumonic plague,; Pneumococcal disease; Poliomyelitis (Polio); Psittacosis; Pthiriasis (crabs; pubic lice infestation); Pustular rash diseases (small pox, monkeypox, cowpox); Q-Fever; Rabies; Ricin poisoning; Rickettsiosis (Rocky Mountain Spotted Fever); Rubella, including congenital rubella (German Measles); Salmonellosis gastroenteritis infection; Scabies infestation; Scombroid; Severe acute respiratory syndrome (SARS); Shigellosis gastroenteritis infection; Smallpox; Methicillin-resistant Staphyloccal infection (MRSA); Staphylococcal food poisoning; Vancomycin intermediate Staphylococcal infection (VISA); Vancomycin resistant Staphylococcal infection (VRSA); Streptococcal disease, Group A; Streptococcal disease, Group B; Streptococcal toxic-shock syndrome (STSS); Primary, secondary, early latent, late latent or congenital syphilis; Tetanus infection (Lock Jaw); Trichonosis; Tuberculosis (TB); Latent tuberculosis (LTBI); Tularemia (rabbit fever); Typhoid fever, Group D; Typhus; Vaginosis; Varicella (chickenpox); Vibrio cholerae infection (Cholera); Vibriosis (Vibrio); Viral hemorrhagic fever (Ebola, Lassa, Marburg); West Nile virus infection; Yellow Fever; Yersenia infection and Zika virus infection.

In some embodiments, the disease is a cancer.

In some embodiments, the cancer is selected in a non-limiting group comprising a bladder cancer, a bone cancer, a brain cancer, a breast cancer, a cancer of the central nervous system, a cancer of the cervix, a cancer of the upper aero digestive tract, a colorectal cancer, an endometrial cancer, a germ cell cancer, a glioblastoma, a Hodgkin lymphoma, a kidney cancer, a laryngeal cancer, a leukaemia, a liver cancer, a lung cancer, a myeloma, a nephroblastoma (Wilms tumor), a neuroblastoma, a non-Hodgkin lymphoma, an oesophageal cancer, an osteosarcoma, an ovarian cancer, a pancreatic cancer, a pleural cancer, a prostate cancer, a retinoblastoma, a skin cancer (including a melanoma), a small intestine cancer, a soft tissue sarcoma, a stomach cancer, a testicular cancer and a thyroid cancer.

In some embodiments, a skilled in the art may understand that ex vivo manipulations and/or therapy may be encompassed within the scope of the instant invention, which would include stem cells and progenitor cells, hematopoietic stem and progenitor cells, induced Pluripotent Stem Cell (iPSC), and adult cells from different species. Without wanting to be bound to a theory, the inventors consider that this is of special interest when a skilled artisan is performing regenerative medicine.

In certain embodiments, the nucleic acids and the nucleic acid vectors encompassed by the instant invention may be employed to engineer animal or plant models, e.g. animal models for preclinical studies, bearing in mind the fundamental ethical principles.

### Methods

The methods disclosed herein may be achieved in vitro, in vivo or ex vivo.

Another aspect of the present invention concerns a method for editing the genome into at least one target cell comprising at least the step of administering to an individual in need thereof the pharmaceutical composition, as defined herein.

In one aspect, the invention concerns a method for preventing and/or treating a disease comprising at least the step of administering to an individual in need thereof the pharmaceutical composition, as defined herein.

In some embodiments, the methods above further comprise a step of providing the individual with a diet deficient in at least one essential amino acid, in particular an amino acid selected in a group comprising histidine (His, H), isoleucine (Ile, I), leucine (Leu, L), Lysine (Lys, K), methionine (Met, M), phenylalanine (Phe, F), threonine (Thr, T), tryptophane (Trp, W) and valine (Val, V).

In certain embodiments, the methods above alternatively comprise a step of administering a compound known to activate the AARE nucleic acid comprised in the regulatory polynucleotide, in particular a compound selected in a group comprising halofuginone, tunicamycin, and the like.

In some embodiments, the disease is selected in a group comprising a genetic disorder, an infectious disease and a cancer.

In some embodiments, the pharmaceutical composition may be administered to an individual in need thereof by any route, i.e. by an oral administration, a topical administration or a parenteral administration, e.g., by injection, including a sub-cutaneous administration, a venous administration, an arterial administration, in intra-muscular administration, an intra-ocular administration, and an intra-auricular administration.

Other modes of administration employ pulmonary formulations, suppositories, and transdermal applications.

In some embodiments, an oral formulation according to the invention includes usual excipients, such as, for example, pharmaceutical grades of mannitol, lactose, starch, magnesium stearate, sodium saccharine, cellulose, magnesium carbonate, and the like.

In some embodiments, an effective amount of said compound is administered to said individual in need thereof.

Within the scope of the instant invention, an “effective amount” refers to the amount of said compound that alone stimulates the desired outcome, i.e. alleviates or eradicates the symptoms of the encompassed disease, in particular a genetic disorder.

It is within the common knowledge of a skilled artisan to determine the effective amount of a nucleic acid for the controlled expression of a nucleic acid encoding a Cas nuclease, or a nucleic acid vector or a delivery particle in order to observe the desired outcome, comprised in a pharmaceutical composition, as defined herein.

Within the scope of the instant invention, the effective amount of the compound to be administered may be determined by a physician or an authorized person skilled in the art and can be suitably adapted within the time course of the treatment.

In certain embodiments, the effective amount to be administered may depend upon a variety of parameters, including the material selected for administration, whether the administration is in single or multiple doses, and the individual’s parameters including age, physical conditions, size, weight, gender, and the severity of the disorder to be treated.

Another aspect of the invention also relates to a method for editing the genome into at least one target cell comprising the steps of:
providing to the target cell
   a nucleic acid for the controlled expression of a nucleic acid encoding a Cas nuclease, as disclosed herein;
   a guide DNA or RNA, which is specific of a genomic target nucleic acid to be edited;
   a donor nucleic acid comprising a nucleic acid intended to replace the target genomic nucleic acid;
inducing the expression of the Cas nuclease.

Upon induction of the Cas nuclease, the Cas nuclease will promote single strand or double strand break(s) in the genomic target nucleic acid, with the assistance of the guide DNA or RNA, and on the donor nucleic acid. Subsequently, the nucleic acid from the donor nucleic acid may be integrated in the genome in the place of the genomic target nucleic acid.

In some embodiments, the induction of the expression of the Cas nuclease may be performed by providing to the target cell a medium that is deficient in at least one essential amino acid or a medium that comprises halofuginone and/or tunicamycin.

In some embodiment, the target nucleic acid has a genetic mutation.

### Kit

In a further aspect, the invention concerns a kit for treating and/or preventing a disease comprising:
a pharmaceutical composition, as defined herein, and
an pharmaceutically active compound.

In some embodiments, the disease is selected in a group comprising a genetic disorder, an infectious disease and a cancer.

Within the scope of the invention, the expression “pharmaceutically active compound” is intended to mean a compound having a benefit towards the prevention and/or the treatment of a given disease.

A skilled artisan understands the term “benefit” as having a positive effect to reduce or alleviate at least one symptom associated with the given disease. By “benefit”, a skilled in the art also understands that the progression of the given disease may be slowed down or stopped.

In some embodiments, the pharmaceutically active compound is an antimicrobial compound, which may be suitably selected by a skilled in the art from the compounds commonly employed to combat an infectious disease, in particular, a bacterial, a fungal or a viral infection.

In certain embodiments, the antimicrobial compound is an antibiotic selected in a group comprising a penicillin, in particular penicillin and amoxicillin; a carbapenem, in particular imipenem; a cephalosporin, in particular cephalexin; an aminoglycoside, in particular gentamicin and tobramycin; a tetracycline, in particular tetracycline and doxycycline; a macrolide, in particular erythromycin and clarithromycin; a quinolone, in particular ciprofloxacin and levofloxacin; and a sulphonamide, in particular sulfamethizole and sulfamethoxazole.

In certain embodiments, the antimicrobial compound is an antiviral agent selected in a non-limiting group comprising a neuraminidase inhibitor; a nucleoside analogue of guanine; a nucleoside analogue of thymidine; a nucleotide reverse transcriptase inhibitor; and a protease inhibitor.

In some embodiments, the pharmaceutically active compound is an anti-cancer compound, which may be suitably selected by a skilled in the art from the compounds commonly employed in chemotherapy.

In certain embodiments, the anti-cancer compound may be selected in a group comprising an alkylating agent, a purine analogue, a pyrimidine analogue, an anthracycline, bleomycin, mytomycin, an inhibitor of topo-isomerase 1, an inhibitor of topo-isomerase 2, a taxan, a monoclonal antibody, a cytokine, an inhibitor of a protein kinase, and the like.

### EXAMPLES

### Example 1: Induction of CAS9 Expression by Essential Amino Acid Starvation

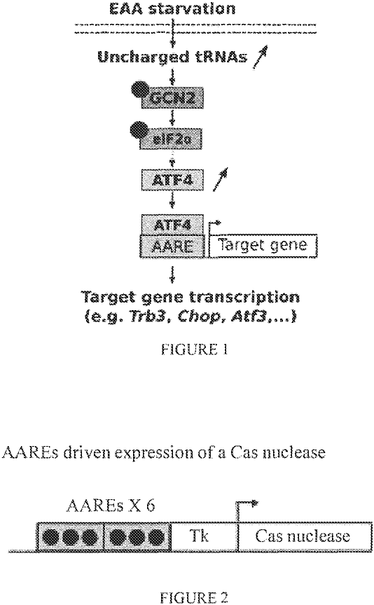

FIG. 1 illustrates the GCN2-eIF2α-ATF4 signaling pathway. In response to EAA starvation, activated GCN2 phosphorylates eIF2α, leading to an up-regulation of the transcription factor ATF4 and its recruitment to AARE sequences to induce target gene expression.

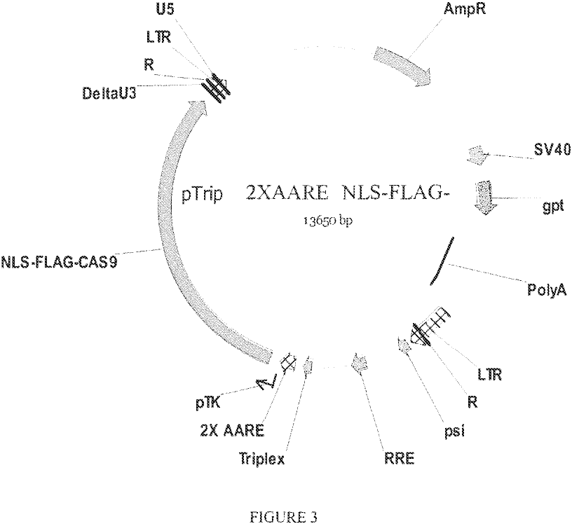

FIG. 2 illustrates the overall strategy for constructing a nucleic acid encoding a Cas nuclease under the regulation of Tk minimal promoter and six copies of the AARE nucleic acid from Trb3 (black spots).

To address CAS9 activity, a cellular model derived from HEK 293T cells bearing a single copy of GFP transgene is used (293T^{GFP} cell line).

This cell line is co-transduced with 2 different lentiviral vectors.

The first one expresses a FLAG tagged version of CAS9 (Shen et al Cell Res. 2013 Apr 2. doi: 10.1038/cr.2013.46; SEQ ID NO: 8) placed under the control of the 2X AARE-TK regulation promoter (SEQ ID NO: 6 and SEQ ID NO: 7).

The second vector expresses a guide RNA specifically targeting the GFP reporter gene (gRNA^{GFP}) under the control of the U6 promoter a RNA polymerase III promoter (Ma, H et al. Mol Ther Nucleic Acids 2014 doi: 10.1038/mtna.2014.12).

Both lentiviral vectors have been constructed in the pTRIP lentiviral backbone (Zennou et al., 2000; Cell 101, 173-185).

The nucleic acid of the pTrip-2XAARE-NLS-FLAG-CAS9 plasmid (SEQ ID NO: 9) is represented in FIG. 3.

As control a third lentiviral vector expressing the FLAG-CAS9 under the control of the EF1a ubiquitous promoter is generated.

The transduced cells are amplified in culture. In absence of induction the 2XAARE-CAS9 cells and the EF1a-CAS9 cells are lysed and the expression of CAS9 is monitored by quantitative RT PCR and the amount of CAS9 protein expression followed by Western blot detection of the FLAG tag. Under such condition only the ubiquitously expressed CA9 is detected and quantified.

Next, the transduced cells are placed in culture in a specific medium depleted in either Leu or Thr. The induction of CAS9 expression in the 2XAARE-CAS9 cells is monitored with time both at the level of mRNA and protein. The optimal treatment period is thus determined.

Finally, when 293T^{GFP} cells are expressing both CAS9 and gRNA^{GFP} expression of GFP in these cells decrease and therefore the percentage of GFP positive cells measured by flow cytometry is an accurate mean to address CAS9 efficiency to knock out the GFP expression.

Thus, without amino acid starvation the percentage of GFP positive cells in FLAG-CAS9 transduced 293T^{GFP} cells remains constant in culture. After optimal induction in culture with deprived AA medium the percentage of GFP cells is dramatically reduced. The overall efficacy is compared to a continuous expression of CAS9 in the cells transduced with EF1a-CAS9.

### Example 2: Induction of CAS9 Expression by Essential Amino Acid Starvation

The promoter 2xAARE contains 6 binding sequences for the transcription factor ATF4, which is rapidly induced in conditions of essential amino acids (EAA) starvation, or other cellular stress such as the stress induced to the endoplasmic reticulum by Tunicamycin (Tu).

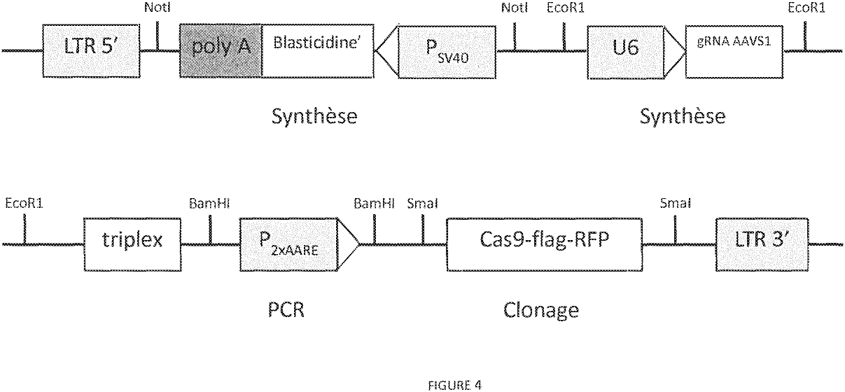

To assess if the expression of the bacterial nuclease Cas9 can be regulated by the promoter 2xAARE, the Cas9 gene of Streptococcus pyogenes (spCas9) fused to a flag tag, an autocatalytic P2A peptide and the red fluorescent protein (RFP) was cloned under the control of the 2xAARE enhancer containing 4 binding sites for ATF4 and the minimal promoter of thymidine kinase gene (TKm) derived from the Herpes simplex virus (HSV; FIG. 4).

This HIV-derived lentiviral vector allows stable expression of the resistance gene Blasticidin for selection of integration events of the vector. A second cassette contains the U6 promoter and the guide RNA AAVS1 (SEQ ID NO: 10) and the CRISPR-associated RNA scaffold, allowing cut below the ATG of the human gene PPP1R12C. A third cassette of expression contains the gene spCas9-flag-RFP under control of the promoter 2xAARE-TKm.

This plasmid was used to produce lentiviral particles according to standard protocol of co-transfection of the vector plasmid, + a plasmid encoding the VSV envelop (pVSV), + a plasmid encoding the HIV Rev gene (pRev), + a plasmid encoding the Gag and Pol genes of HIV (p8.9), in 293T cells. After 48 h, cells supernatants were harvested, ultra-centrifuged for concentration and stored at -80° C. until use. Vector stocks were tittered with real time quantitative PCR to measure viral RNA copies of genomes/ml (Saeed et al.; Mol Ther Nucleic Acids. 2014 Dec 2;3:e213).

To assess whether the expression of spCas9-flag-RFP could be modulated by EAA starvation (medium without Leucin, Leu-) or tunicamycin (Sigma-Aldrich®), 293T cells were transduced with 100 vRNAc per cell and then selected with blasticidin at 2 µg/ml (Sigma-Aldrich®). Non-transduced 293T cells all died, while transduced cells grew normally indicating that they all contained at least one copy of the vector pTRIP blast_U6 AAVS1_2xAARE-Cas9-flag-RFP.

This population, called 293-C9, was expanded and used for further experiments. Cells were plated in 24 well plates (105 cells/well) and the expression of the gene Cas9-flag-RFP was induced either with culture medium with 10% serum depleted from Leucine (DMEM Leu-), with tunicamycin at 0.5 µg/ml (DMEM complete + Tu) or control medium (DMEM complete).

Cells were collected at several times after induction (4 h, 8 h, 24 h) as well as after 24 h (i.e. 48 h after induction) and 48 h (i.e. 72 h after induction) after removing the inducing medium and replacing it by a complete medium. Cells collected at diverse time points were pelleted and lysed for protein purification (Tris-HCl 0.05 M; SDS 0.5%; 1 mM DTT; pH 8.0 with anti-proteases).

Proteins concentration were measured using Bradford test and 30 µg of lysate mixed with loading buffer and beta-mercaptoethanol and heated at 95° C. for 5 minutes, was loaded on a denaturing SDS 10% polyacrylamide gel. Proteins were separated with electrophoresis. Migration was monitored with colored ladder.

Proteins on the gel were transferred to a nitrocellulose membrane through semi-dry transfer and membrane was used for immunoblot using an anti-FLAG M2 MAB (Sigma-Aldrich®), then detected with a secondary anti-mouse antibody coupled to HRP. Peroxydase activity was revealed with Chemiluminescent HRP substrate (Luminata crescendo -Millipore®) and pictured with a chemiluminence detector Fusion FX7 (Vilber®).

The density of the detected bands of SPCas9-Flag-RFP of 193 kDa was quantified with the software of the Fusion FX7 detector (Vilber®). The level of beta-actin in each sample was also measured in the same way but using an anti-beta actin primary antibody. The density of the bands corresponding to Cas9-flag-RFP was normalized with the density of beta-actin. As these experiments were performed on different gels and to homogenize data, the band of highest density (in both cases after 24 h induction) was considered as 100% of expression and the values of the other bands of lower density were compared as percentage of the most intense reference in each gel.

As can be seen in FIG. 5, at non-induced basal level of expression of 2xAARE-Tkm, the Cas9-flag-RFP fusion remains undetectable. However, the expression of the fusion is rapidly induced upon addition of Leu- medium to the cells (plain line) or upon addition of a complete medium containing Tu (dashed line). Upon removal of inducing medium (at 24 h) and its replacement with complete medium, the expression of the protein Cas9-flag-RFP decreased progressively (see at 48 h and 72 h). This indicates that the promoter 2xAARE-Tkm allows the control of Cas9 expression with EAA starvation or through induction of ER stress. To assess whether the induction of the protein Cas9-flag-RFP allows cutting, (i.e. performing double strand breaks), at the AAVS1 genomic location in the genome, 293-C9 cells were cultured for 24 h either with DMEM complete or DMEM Leu-. Cells were harvested after 24 h, pelleted and genomic DNA was purified using a DNA easy Kit (Quiagen®). A PCR was performed with primers hybridizing on 5′ (SEQ ID NO: 11) and on 3′(SEQ ID NO: 12) of the AAVS1 cutting site. The PCR product of 540 bp was purified and sequenced and was confirmed to be the targeted one.

To further assess whether Cas9 had cut we performed a T7 nuclease test (New England Biolabs®). The PCRs amplifying the AAVS1 band from purified genomic DNA of 293-C9 not induced and induced were denatured and slowly re-hybridized following provider’s guidelines www.neb.com/protocols/2014/08/11/determining-genome-targeting-efficiency-using-t7-endonuclease-i).

Cas9-induced double strand breaks are repaired by the cell machinery and produces insertions and deletions (indels) at the site of cutting, thus re-hybridization of PCR bands obtained from a population of cells containing a mixture of different indels produces DNA fragments containing mismatches. These are cut by the T7 nuclease releasing smaller bands of DNA.

It could be observed that induction of Cas9-flag-RFP expression with DMEM Leu- for 24 h in 293-C9 cells produces smaller bands at 250 bp corresponding to the cutting site AAVS1 placed in the center of the PCR band representing about 20 % of the total DNA. In non-induced 293-C9 cells such bands are not apparent, indicating that the AAVS1 site remains uncut until induction of the gene Cas9-flag-RFP.

Therefore, the system for the controlled expression of Cas9, on a mode ON/OFF, as disclosed herein provides a tool for safely editing the genome. Indeed, in the absence of induction, the absence of any detectable leakage of the expression system provides a safety feature for preventing any unwanted genome editing.

Contrarily, in the presence of induction, the rapid expression may be shut down as soon as the removal of induction is effective.

Two functional tests were then performed to integrate a donor DNA (Do) at the AAVS1 site.

In the first one, 293T cells were transfected (Ca²⁺ phosphate method) with the plasmid ‘pTRIP blast_U6 AAVS1_2xAARE-Cas9-flag-RFP’ as well as with the donor plasmid containing a cassette ‘AAVS1 cut site-GFP-p2a-Puromycin_AAVS1 cut site’, accepting cuts with Cas9 + gRNA AAVS1 on 5′ and 3′ of the GFP-p2a- Puromycin gene.

The chosen AAVS1 site targeted by the guide RNA in the genome of 293T cells includes the ATG start codon of the gene PPP1R12C. When targeted, it will allow insertion of the released GFP-p2a-Puromycin cassette in the place of the exon 1 of PPP1R12C and subsequently expression by the PPP1R12C promoter. In this case, recombinant cells express GFP and become resistant to puromycin allowing their selection and to count the clones corresponding to integration events.

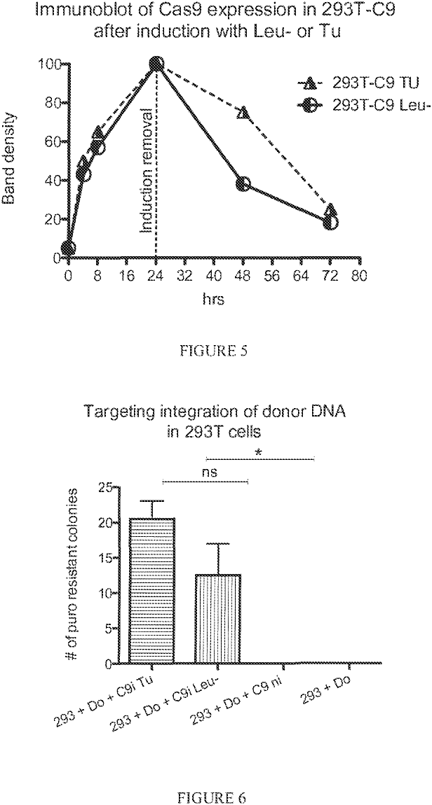

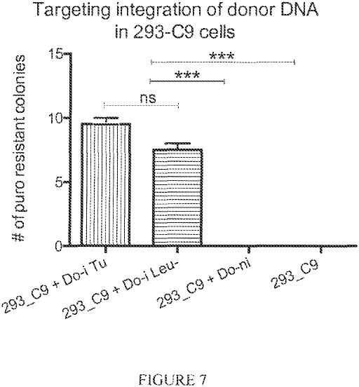

As shown in FIG. 6, the donor construct gives resistant clones to puromycine only when both plasmids ‘pTRIP blast_U6 AAVS 1_2xAARE-Cas9-flag-RFP’ (C9) and donor ‘pAAVS1 cut site-GFP-p2a-Puromycin_AAVS1 cut site’ (Do) are provided together with 2xAARE induction (i) either with a Leucine deprived medium (i Leu-) or a Tunicamycin-containing medium (i Tu), but not with complete medium (ni). This indicates that Cas9 activity for targeted integration requires induction of the promoter 2xAARE.

This experiment was replicated in 293-C9 cells transfected with the Donor plasmid containing -GFP-p2a-Puromycin flanked by 2 AAVS1 cut sites (Do). In such a plasmid pAAVS 1-guided Cas9 activity will release the GFP-p2a-Puromycin sequence

As can be seen in FIG. 5, when 293-C9 cells are transfected with Donor plasmid (Do), no puromycin resistant colonies are produced in absence of induction (ni). In contrast, when 293-C9 cells are transfected with the donor plasmid (Do) and induced in the presence of Tunicamycin or a Leucine-deprived medium, these cells produced puromycin resistant colonies in both conditions.

This further confirms that, upon induction of Cas9 expression, the Cas9 nuclease, when guided to AAVS1 cut sites, is efficient to generate double strand breaks, in both (1) the donor plasmid, resulting in the release the donor cassette, and (2) in the AAVS1 site in the genomic DNA, resulting in the integration of the donor cassette.

The examples above provide convincing experimental data showing that the system for the controlled expression of a Cas9 nuclease, which expression is based upon AAREs, may be finely tuned by the induction conditions.

Indeed, in the absence of induction, no detectable expression is observed, which means that leakage is not observed.

In addition, genome editing may only be observed upon induction, and may be shut down rapidly upon removal of the induction conditions.

Therefore, because this system may be turned on and turned off very precisely, this system offers a safe tool for providing genome editing and hence gene therapy in individuals in need thereof.

### NUCLEIC SEQUENCES DISLOSED IN THE INVENTION

The Table 1 below discloses the nucleic acid sequences used herein:

| SEQ ID NO: | Type | Comments |
|---|---|---|
| 1 | Nucleic acid | AARE sequence from the TRIB3 gene |
| 2 | Nucleic acid | AARE sequence from the CHOP gene |
| 3 | Nucleic acid | AARE sequence from the ASNS gene |
| 4 | Nucleic acid | AARE sequence from the ATF3 gene |
| 5 | Nucleic acid | AARE sequence from the SNAT2 gene |
| 6 | Nucleic acid | Thymidine kinase minimal promoter |
| 7 | Nucleic acid | 2XAARE nucleic acid |
| 8 | Nucleic acid | NLS-FLAG CAS9 nucleic acid |
| 9 | Nucleic acid | pTRIP 2XAARE- NLS-FLAG CAS9 nucleic acid |
| 10 | Nucleic acid | guide RNA AAVS1 |
| 11 | Nucleic acid | 5′ primer |
| 12 | Nucleic acid | 3′ primer |

AARE sequence from the TRIB3 gene: SEQ ID NO: 1

cggtttgcatcacccg

AARE sequence from the CHOP gene: SEQ ID NO: 2

aacattgcatcatccc

AARE sequence from the ASNS gene: SEQ ID NO: 3

gaagtttcatcatgcc

AARE sequence from the ATF3 gene: SEQ ID NO: 4

agcgttgcatcacccc

AARE sequence from the SNAT2 gene: SEQ ID NO: 5

gatattgcatcagttt

Thymidine kinase minimal promoter nucleic acid: SEQ ID NO: 6

cgaggtccacttcgcatattaaggtgacgcgtgtggcctcgaacaccgag
cgaccctgcagcgacccgcttaacagcgtcaacagcgtgccgca

2XAARE nucleic acid: SEQ ID NO: 7

gattagctccggtttgcatcacccggaccgggggattagctccggtttgc
atcacccggaccgggggattagctccggtttgcatcacccggaccggggg
ccgggcgcgtgctagcgattagctccggtttgcatcacccggaccggggg
attagctccggtttgcatcacccggaccgggggattagctccggtttgca
tcacccggaccgggg

NLS-FLAG CAS9 nucleic acid: SEQ ID NO: 8

atgggacctaagaaaaagaggaaggtgcctaagaaaaagaggaaggtgcc
taagaaaaagaggaaggtggcggccgctgactacaaggatgacgacgata
aatctagagacaagaaatactctattggactggatatcgggacaaactcc
gttggctgggccgtcataaccgacgagtataaggtgccaagcaagaaatt
caaggtgctgggtaatactgaccgccattcaatcaagaagaacctgatcg
gagcactcctcttcgactccggtgaaaccgctgaagctactcggctgaag
cggaccgcaaggcggagatacacccgccgcaagaatcggatatgttatct
gcaagagatctttagcaacgaaatggctaaggtggacgactccttctttc
accgcctggaagagagctttctggtggaggaggataagaaacacgagagg
caccctatattcggaaatatcgtggatgaggtggcttaccatgaaaagta
tcctacaatctaccatctgaggaagaagctggtggacagcaccgataaag
cagacctgaggctcatctatctggccctggctcatatgataaagtttaga
ggacactttctgatcgagggcgacctgaatcccgataattccgatgtgga
taaactcttcattcaactggtgcagacatataaccaactgttcgaggaga
atcccataaacgcttctggtgtggatgccaaggctattctgtccgctcgg
ctgtccaagtcacgcagactggagaatctgattgcccaactgccaggaga
aaagaagaacggcctgtttgggaacctcatcgccctgagcctgggcctga
cacctaacttcaagtccaattttgatctggccgaagatgctaaactccag
ctctccaaggacacctatgacgatgatctggacaacctgctcgcacagat
aggcgaccagtacgccgatctctttctggctgctaagaatctctccgacg
ccattctgctgagcgacatactccgggtcaacactgagatcaccaaagca
cctctgagcgcctccatgataaaacgctatgatgaacaccatcaagacct
gactctgctcaaagccctcgtgaggcaacagctgccagagaagtacaaag
agatattcttcgaccagagcaagaatggatatgccggatacatcgatggc
ggagcatcacaggaagaattttacaagttcatcaaaccaatcctcgagaa
gatggacggtactgaagagctgctggtgaagctgaacagggaggacctgc
tgaggaagcagaggacctttgataatggctccattccacatcagatacac
ctgggagagctgcatgcaatcctccgcaggcaggaggatttctatccttt
cctgaaggataaccgggagaagatagagaagatcctgaccttcaggatcc
cttattacgtcggccctctggctagaggcaactcccgcttcgcttggatg
accaggaaatctgaggagacaattactccttggaacttcgaagaggtcgt
ggataagggcgcaagcgcccagtcattcatcgaacggatgaccaatttcg
ataagaacctgcccaacgagaaggtcctgcccaaacattcactcctgtac
gagtatttcaccgtctataacgagctgactaaagtgaagtacgtgaccga
gggcatgaggaagcctgccttcctgtccggagagcagaagaaggctatcg
ttgatctgctcttcaagactaatagaaaggtgacagtgaagcagctcaag
gaggattactttaagaagatcgaatgctttgactcagtggaaatctctgg
cgtggaggaccgctttaatgccagcctgggcacttaccatgatctgctga
agataatcaaagacaaagatttcctcgataatgaggagaacgaggacatc
ctggaagatatcgtgctgaccctgactctgttcgaggatagagagatgat
cgaagagcgcctgaagacctatgcccatctgtttgacgataaagtcatga
aacagctcaagcggcggcgctacactgggtggggtagactctccaggaaa
ctcataaacggcatccgcgacaaacagagcggaaagaccatcctggattt
cctgaaatccgacggattcgctaacaggaacttcatgcaactgattcacg
atgactctctgacatttaaagaggacatccagaaggcacaggtgagcggt
caaggcgacagcctgcacgagcacatcgccaacctcgctggatcacccgc
cataaagaagggaatactgcagacagtcaaggtcgtggacgaactcgtca
aagtgatgggtcggcacaagccagagaatatcgttatcgaaatggcaagg
gagaaccaaaccacccagaagggccagaagaactctcgggaacggatgaa
aagaatcgaagagggaattaaggagctgggatctcagatactgaaggagc
accctgtggagaatacacagctccagaacgagaaactctacctgtactac
ctccagaacgggcgggacatgtacgttgaccaggaactcgacatcaaccg
gctgtccgattatgacgtggaccatattgttccacagtccttcctcaaag
atgactccattgacaacaaggtgctgaccagatccgataagaatcgcggt
aagtctgacaatgttccatcagaagaggtggtcaagaagatgaagaatta
ctggcggcagctcctcaacgccaaactgatcacccagcggaagtttgaca
atctgactaaggcagaaagaggaggtctgagcgaactcgacaaggccggc
tttattaagaggcaactggtcgaaacacgccagattaccaaacacgtggc
acaaatcctcgactctaggatgaacactaagtacgatgagaacgataagc
tgatcagggaagtgaaagtgataactctgaagagcaagctggtgtctgac
ttccggaaggactttcaattctacaaagttcgcgaaataaacaattacca
tcatgctcacgatgcctatctcaatgctgtcgttggcaccgccctgatca
agaaataccctaaactggagtctgagttcgtgtacggtgactataaagtc
tacgatgtgaggaagatgatagcaaagtctgagcaagagattggcaaagc
caccgccaagtacttcttctactctaatatcatgaatttctttaagactg
agataaccctggctaacggcgaaatccggaagcgcccactgatcgaaaca
aacggagaaacaggagaaatcgtgtgggataaaggcagggacttcgcaac
tgtgcggaaggtgctgtccatgccacaagtcaatatcgtgaagaagaccg
aagtgcagaccggcggattctcaaaggagagcatcctgccaaagcggaac
tctgacaagctgatcgccaggaagaaagattgggacccaaagaagtatgg
cggtttcgattcccctacagtggcttattccgttctggtcgtggcaaaag
tggagaaaggcaagtccaagaaactcaagtctgttaaggagctgctcgga
attactattatggagagatccagcttcgagaagaatccaatcgatttcct
ggaagctaagggctataaagaagtgaagaaagatctcatcatcaaactgc
ccaagtactctctctttgagctggagaatggtaggaagcggatgctggcc
tccgccggagagctgcagaaaggaaacgagctggctctgccctccaaata
cgtgaacttcctgtatctggcctcccactacgagaaactcaaaggtagcc
ctgaagacaatgagcagaagcaactctttgttgagcaacataaacactac
ctggacgaaatcattgaacagattagcgagttcagcaagcgggttattct
ggccgatgcaaacctcgataaagtgctgagcgcatataataagcacaggg
acaagccaattcgcgaacaagcagagaatattatccacctctttactctg
actaatctgggcgctcctgctgccttcaagtatttcgatacaactattga
caggaagcggtacacctctaccaaagaagttctcgatgccaccctgatac
accagtcaattaccggactgtacgagactcgcatcgacctgtctcagctc
ggcggcgactag

Nucleic acid of the pTrip-2XAARE-NLS-FLAG-CAS9 plasmid: SEQ ID NO: 9

ccagatcctctacgccggacgcatcgtggccggcatcaccggcgccacag
gtgcggttgctggcgcctatatcgccgacatcaccgatggggaagatcgg
gctcgccacttcgggctcatgagcgcttgtttcggcgtgggtatggtggc
aggccccgtggccgggggactgttgggcgccatctccttgcatgcaccat
tccttgcggcggcggtgctcaacggcctcaacctactactgggctgcttc
ctaatgcaggagtcgcataagggagagcgtcgaatggtgcactctcagta
caatctgctctgatgccgcatagttaagccagccccgacacccgccaaca
cccgctgacgcgccctgacgggcttgtctgctcccggcatccgcttacag
acaagctgtgaccgtctccgggagctgcatgtgtcagaggttttcaccgt
catcaccgaaacgcgcgagacgaaagggcctcgtgatacgcctattttta
taggttaatgtcatgataataatggtttcttagacgtcaggtggcacttt
tcggggaaatgtgcgcggaacccctatttgtttatttttctaaatacatt
caaatatgtatccgctcatgagacaataaccctgataaatgcttcaataa
tattgaaaaaggaagagtatgagtattcaacatttccgtgtcgcccttat
tcccttttttgcggcattttgccttcctgtttttgctcacccagaaacgc
tggtgaaagtaaaagatgctgaagatcagttgggtgcacgagtgggttac
atcgaactggatctcaacagcggtaagatccttgagagttttcgccccga
agaacgttttccaatgatgagcacttttaaagttctgctatgtggcgcgg
tattatcccgtattgacgccgggcaagagcaactcggtcgccgcatacac
tattctcagaatgacttggttgagtactcaccagtcacagaaaagcatct
tacggatggcatgacagtaagagaattatgcagtgctgccataaccatga
gtgataacactgcggccaacttacttctgacaacgatcggaggaccgaag
gagctaaccgcttttttgcacaacatgggggatcatgtaactcgccttga
tcgttgggaaccggagctgaatgaagccataccaaacgacgagcgtgaca
ccacgatgcctgtagcaatggcaacaacgttgcgcaaactattaactggc
gaactacttactctagcttcccggcaacaattaatagactggatggaggc
ggataaagttgcaggaccacttctgcgctcggcccttccggctggctggt
ttattgctgataaatctggagccggtgagcgtgggtctcgcggtatcatt
gcagcactggggccagatggtaagccctcccgtatcgtagttatctacac
gacggggagtcaggcaactatggatgaacgaaatagacagatcgctgaga
taggtgcctcactgattaagcattggtaactgtcagaccaagtttactca
tatatactttagattgatttaaaacttcatttttaatttaaaaggatcta
ggtgaagatcctttttgataatctcatgaccaaaatcccttaacgtgagt
tttcgttccactgagcgtcagaccccgtagaaaagatcaaaggatcttct
tgagatcctttttttctgcgcgtaatctgctgcttgcaaacaaaaaaacc
accgctaccagcggtggtttgtttgccggatcaagagctaccaactcttt
ttccgaaggtaactggcttcagcagagcgcagataccaaatactgtcctt
ctagtgtagccgtagttaggccaccacttcaagaactctgtagcaccgcc
tacatacctcgctctgctaatcctgttaccagtggctgctgccagtggcg
ataagtcgtgtcttaccgggttggactcaagacgatagttaccggataag
gcgcagcggtcgggctgaacggggggttcgtgcacacagcccagcttgga
gcgaacgacctacaccgaactgagatacctacagcgtgagcattgagaaa
gcgccacgcttcccgaagggagaaaggcggacaggtatccggtaagcggc
agggtcggaacaggagagcgcacgagggagcttccagggggaaacgcctg
gtatctttatagtcctgtcgggtttcgccacctctgacttgagcgtcgat
ttttgtgatgctcgtcaggggggcggagcctatggaaaaacgccagcaac
gcggcctttttacggttcctggccttttgctggccttttgctcacatgtt
ctttcctgcgttatcccctgattctgtggataaccgtattaccgcctttg
agtgagctgataccgctcgccgcagccgaacgaccgagcgcagcgagtca
gtgagcgaggaagcggaagagcgcccaatacgcaaaccgcctctccccgc
gcgttggccgattcattaatgcagctgtggaatgtgtgtcagttagggtg
tggaaagtccccaggctccccagcaggcagaagtatgcaaagcatgcatc
tcaattagtcagcaaccaggtgtggaaagtccccaggctccccagcaggc
agaagtatgcaaagcatgcatctcaattagtcagcaaccatagtcccgcc
cctaactccgcccatcccgcccctaactccgcccagttccgcccattctc
cgccccatggctgactaattttttttatttatgcagaggccgaggccgcc
tcggcctctgagctattccagaagtagtgaggaggcttttttggaggcct
aggcttttgcaaaaagcttggacacaagacaggcttgcgagatatgtttg
agaataccactttatcccgcgtcagggagaggcagtgcgtaaaaagacgc
ggactcatgtgaaatactggtttttagtgcgccagatctctataatctcg
cgcaacctattttcccctcgaacactttttaagccgtagataaacaggct
gggacacttcacatgagcgaaaaatacatcgtcacctgggacatgttgca
gatccatgcacgtaaactcgcaagccgactgatgccttctgaacaatgga
aaggcattattgccgtaagccgtggcggtctgtaccgggtgcgttactgg
cgcgtgaactgggtattcgtcatgtcgataccgtttgtatttccagctac
gatcacgacaaccagcgcgagcttaaagtgctgaaacgcgcagaaggcga
tggcgaaggcttcatcgttattgatgacctggtggataccggtggtactg
cggttgcgattcgtgaaatgtatccaaaagcgcactttgtcaccatcttc
gcaaaaccggctggtcgtccgctggttgatgactatgttgttgatatccc
gcaagatacctggattgaacagccgtgggatatgggcgtcgtattcgtcc
cgccaatctccggtcgctaatcttttcaacgcctggcactgccgggcgtt
gttctttttaacttcaggcgggttacaatagtttccagtaagtattctgg
aggctgcatccatgacacaggcaaacctgagcgaaaccctgttcaaaccc
cgctttaaacatcctgaaacctcgacgctagtccgccgctttaatcacgg
cgcacaaccgcctgtgcagtcggcccttgatggtaaaaccatccctcact
ggtatcgcatgattaaccgtctgatgtggatctggcgcggcattgaccca
cgcgaaatcctcgacgtccaggcacgtattgtgatgagcgatgccgaacg
taccgacgatgatttatacgatacggtgattggctaccgtggcggcaact
ggatttatgagtgggccccggatctttgtgaaggaaccttacttctgtgg
tgtgacataattggacaaactacctacagagatttaaagctctaaggtaa
atataaaatttttaagtgtataatgtgttaaactactgattctaattgtt
tgtgtattttagattccaacctatggaactgatgaatgggagcagtggtg
gaatgcctttaatgaggaaaacctgttttgctcagaagaaatgccatcta
gtgatgatgaggctactgctgactctcaacattctactcctccaaaaaag
aagagaaaggtagaagaccccaaggactttccttcagaattgctaagttt
tttgagtcatgctgtgtttagtaatagaactcttgcttgctttgctattt
acaccacaaaggaaaaagctgcactgctatacaagaaaattatggaaaaa
tattctgtaacctttataagtaggcataacagttataatcataacatact
gttttttcttactccacacaggcatagagtgtctgctattaataactatg
ctcaaaaattgtgtacctttagctttttaatttgtaaaggggttaataag
gaatatttgatgtatagtgccttgactagagatcataatcagccatacca
catttgtagaggttttacttgctttaaaaaacctcccacacctccccctg
aacctgaaacataaaatgaatgcaattgttgttgttaacttgtttattgc
agcttataatggttacaaataaagcaatagcatcacaaatttcacaaata
aagcatttttttcactgcattctagttgtggtttgtccaaactcatcaat
gtatcttatcatgtctggatcaactggataactcaagctaaccaaaatca
tcccaaacttcccaccccataccctattaccactgccaattacctagtgg
tttcatttactctaaacctgtgattcctctgaattattttcattttaaag
aaattgtatttgttaaatatgtactacaaacttagtagttggaagggcta
attcactcccaaagaagacaagatatccttgatctgtggatctaccacac
acaaggctacttccctgattagcagaactacacaccagggccaggggtca
gatatccactgacctttggatggtgctacaagctagtaccagttgagcca
gataaggtagaagaggccaataaaggagagaacaccagcttgttacaccc
tgtgagcctgcatgggatggatgacccggagagagaagtgttagagtgga
ggtttgacagccgcctagcatttcatcacgtggcccgagagctgcatccg
gagtacttcaagaactgctgatatcgagcttgctacaagggactttccgc
tggggactttccagggaggcgtggcctgggcgggactggggagtggcgag
ccctcagatcctgcatataagcagctgctttttgcctgtactgggtctct
ctggttagaccagatctgagcctgggagctctctggctaactagggaacc
cactgcttaagcctcaataaagcttgccttgagtgcttcaagtagtgtgt
gcccgtctgttgtgtgactctggtaactagagatccctcagaccctttta
gtcagtgtggaaaatctctagcagtggcgcccgaacagggacttgaaagc
gaaagggaaaccagaggagctctctcgacgcaggactcggcttgctgaag
cgcgcacggcaagaggcgaggggcggcgactggtgagtacgccaaaaatt
ttgactagcggaggctagaaggagagagatgggtgcgagagcgtcagtat
taagcgggggagaattagatcgcgatgggaaaaaattcggttaaggccag
ggggaaagaaaaaatataaattaaaacatatagtatgggcaagcagggag
ctagaacgattcgcagttaatcctggcctgttagaaacatcagaaggctg
tagacaaatactgggacagctacaaccatcccttcagacaggatcagaag
aacttagatcattatataatacagtagcaaccctctattgtgtgcatcaa
aggatagagataaaagacaccaaggaagctttagacaagatagaggaaga
gcaaaacaaaagtaagaccaccgcacagcaagcggccgctgatcttcaga
cctggaggaggagatatgagggacaattggagaagtgaattatataaata
taaagtagtaaaaattgaaccattaggagtagcacccaccaaggcaaaga
gaagagtggtgcagagagaaaaaagagcagtgggaataggagctttgttc
cttgggttcttgggagcagcaggaagcactatgggcgcagcgtcaatgac
gctgacggtacaggccagacaattattgtctggtatagtgcagcagcaga
acaatttgctgagggctattgaggcgcaacagcatctgttgcaactcaca
gtctggggcatcaagcagctccaggcaagaatcctggctgtggaaagata
cctaaaggatcaacagctcctggggatttggggttgctctggaaaactca
tttgcaccactgctgtgccttggaatgctagttggagtaataaatctctg
gaacagatttggaatcacacgacctggatggagtgggacagagaaattaa
caattacacaagcttaatacactccttaattgaagaatcgcaaaaccagc
aagaaaagaatgaacaagaattattggaattagataaatgggcaagtttg
tggaattggtttaacataacaaattggctgtggtatataaaattattcat
aatgatagtaggaggcttggtaggtttaagaatagtttttgctgtacttt
ctatagtgaatagagttaggcagggatattcaccattatcgtttcagacc
cacctcccaaccccgaggggacccgacaggcccgaaggaatagaagaaga
aggtggagagagagacagagacagatccattcgattagtgaacggatctc
gacggtatcgccgaattcacaaatggcagtattcatccacaattttaaaa
gaaaaggggggattggggggtacagtgcaggggaaagaatagtagacata
atagcaacagacatacaaactaaagaattacaaaaacaaattacaaaaat
tcaaaattttcgggtttattacagggacagcagagatccactttggctga
tacgcggatctacgcgtcaagtttgtacaaaaaagcaggctccgcggccg
cccccttcaccggtaccgattagctccggtttgcatcacccggaccgggg
gattagctccggtttgcatcacccggaccgggggattagctccggtttgc
atcacccggaccgggggccgggcgcgtgctagcgattagctccggtttgc
atcacccggaccgggggattagctccggtttgcatcacccggaccggggg
attagctccggtttgcatcacccggaccggggactcgaggtccacttcgc
atattaaggtgacgcgtgtggcctcgaacaccgagcgaccctgcagcgac
ccgcttaacagcgtcaacagcgtgccgcaagcttgaattctgatcagcat
tccggtactgttggtaaagccaccatgggacctaagaaaaagaggaaggt
gcctaagaaaaagaggaaggtgcctaagaaaaagaggaaggtggcggccg
ctgactacaaggatgacgacgataaatctagagacaagaaatactctatt
ggactggatatcgggacaaactccgttggctgggccgtcataaccgacga
gtataaggtgccaagcaagaaattcaaggtgctgggtaatactgaccgcc
attcaatcaagaagaacctgatcggagcactcctcttcgactccggtgaa
accgctgaagctactcggctgaagcggaccgcaaggcggagatacacccg
ccgcaagaatcggatatgttatctgcaagagatctttagcaacgaaatgg
ctaaggtggacgactccttctttcaccgcctggaagagagctttctggtg
gaggaggataagaaacacgagaggcaccctatattcggaaatatcgtgga
tgaggtggcttaccatgaaaagtatcctacaatctaccatctgaggaaga
agctggtggacagcaccgataaagcagacctgaggctcatctatctggcc
ctggctcatatgataaagtttagaggacactttctgatcgagggcgacct
gaatcccgataattccgatgtggataaactcttcattcaactggtgcaga
catataaccaactgttcgaggagaatcccataaacgcttctggtgtggat
gccaaggctattctgtccgctcggctgtccaagtcacgcagactggagaa
tctgattgcccaactgccaggagaaaagaagaacggcctgtttgggaacc
tcatcgccctgagcctgggcctgacacctaacttcaagtccaattttgat
ctggccgaagatgctaaactccagctctccaaggacacctatgacgatga
tctggacaacctgctcgcacagataggcgaccagtacgccgatctctttc
tggctgctaagaatctctccgacgccattctgctgagcgacatactccgg
gtcaacactgagatcaccaaagcacctctgagcgcctccatgataaaacg
ctatgatgaacaccatcaagacctgactctgctcaaagccctcgtgaggc
aacagctgccagagaagtacaaagagatattcttcgaccagagcaagaat
ggatatgccggatacatcgatggcggagcatcacaggaagaattttacaa
gttcatcaaaccaatcctcgagaagatggacggtactgaagagctgctgg
tgaagctgaacagggaggacctgctgaggaagcagaggacctttgataat
ggctccattccacatcagatacacctgggagagctgcatgcaatcctccg
caggcaggaggatttctatcctttcctgaaggataaccgggagaagatag
agaagatcctgaccttcaggatcccttattacgtcggccctctggctaga
ggcaactcccgcttcgcttggatgaccaggaaatctgaggagacaattac
tccttggaacttcgaagaggtcgtggataagggcgcaagcgcccagtcat
tcatcgaacggatgaccaatttcgataagaacctgcccaacgagaaggtc
ctgcccaaacattcactcctgtacgagtatttcaccgtctataacgagct
gactaaagtgaagtacgtgaccgagggcatgaggaagcctgccttcctgt
ccggagagcagaagaaggctatcgttgatctgctcttcaagactaataga
aaggtgacagtgaagcagctcaaggaggattactttaagaagatcgaatg
ctttgactcagtggaaatctctggcgtggaggaccgctttaatgccagcc
tgggcacttaccatgatctgctgaagataatcaaagacaaagatttcctc
gataatgaggagaacgaggacatcctggaagatatcgtgctgaccctgac
tctgttcgaggatagagagatgatcgaagagcgcctgaagacctatgccc
atctgtttgacgataaagtcatgaaacagctcaagcggcggcgctacact
gggtggggtagactctccaggaaactcataaacggcatccgcgacaaaca
gagcggaaagaccatcctggatttcctgaaatccgacggattcgctaaca
ggaacttcatgcaactgattcacgatgactctctgacatttaaagaggac
atccagaaggcacaggtgagcggtcaaggcgacagcctgcacgagcacat
cgccaacctcgctggatcacccgccataaagaagggaatactgcagacag
tcaaggtcgtggacgaactcgtcaaagtgatgggtcggcacaagccagag
aatatcgttatcgaaatggcaagggagaaccaaaccacccagaagggcca
gaagaactctcgggaacggatgaaaagaatcgaagagggaattaaggagc
tgggatctcagatactgaaggagcaccctgtggagaatacacagctccag
aacgagaaactctacctgtactacctccagaacgggcgggacatgtacgt
tgaccaggaactcgacatcaaccggctgtccgattatgacgtggaccata
ttgttccacagtccttcctcaaagatgactccattgacaacaaggtgctg
accagatccgataagaatcgcggtaagtctgacaatgttccatcagaaga
ggtggtcaagaagatgaagaattactggcggcagctcctcaacgccaaac
tgatcacccagcggaagtttgacaatctgactaaggcagaaagaggaggt
ctgagcgaactcgacaaggccggctttattaagaggcaactggtcgaaac
acgccagattaccaaacacgtggcacaaatcctcgactctaggatgaaca
ctaagtacgatgagaacgataagctgatcagggaagtgaaagtgataact
ctgaagagcaagctggtgtctgacttccggaaggactttcaattctacaa
agttcgcgaaataaacaattaccatcatgctcacgatgcctatctcaatg
ctgtcgttggcaccgccctgatcaagaaataccctaaactggagtctgag
ttcgtgtacggtgactataaagtctacgatgtgaggaagatgatagcaaa
gtctgagcaagagattggcaaagccaccgccaagtacttcttctactcta
atatcatgaatttctttaagactgagataaccctggctaacggcgaaatc
cggaagcgcccactgatcgaaacaaacggagaaacaggagaaatcgtgtg
ggataaaggcagggacttcgcaactgtgcggaaggtgctgtccatgccac
aagtcaatatcgtgaagaagaccgaagtgcagaccggcggattctcaaag
gagagcatcctgccaaagcggaactctgacaagctgatcgccaggaagaa
agattgggacccaaagaagtatggcggtttcgattcccctacagtggctt
attccgttctggtcgtggcaaaagtggagaaaggcaagtccaagaaactc
aagtctgttaaggagctgctcggaattactattatggagagatccagctt
cgagaagaatccaatcgatttcctggaagctaagggctataaagaagtga
agaaagatctcatcatcaaactgcccaagtactctctctttgagctggag
aatggtaggaagcggatgctggcctccgccggagagctgcagaaaggaaa
cgagctggctctgccctccaaatacgtgaacttcctgtatctggcctccc
actacgagaaactcaaaggtagccctgaagacaatgagcagaagcaactc
tttgttgagcaacataaacactacctggacgaaatcattgaacagattag
cgagttcagcaagcgggttattctggccgatgcaaacctcgataaagtgc
tgagcgcatataataagcacagggacaagccaattcgcgaacaagcagag
aatattatccacctctttactctgactaatctgggcgctcctgctgcctt
caagtatttcgatacaactattgacaggaagcggtacacctctaccaaag
aagttctcgatgccaccctgatacaccagtcaattaccggactgtacgag
actcgcatcgacctgtctcagctcggcggcgactagtaaagcggccgggc
tcgagtctagaaagggtgggcgcgccgacccagctttcttgtacaaagtg
gctcgacggtacctttaagaccaatgacttacaaggcagctgtagatctt
agccactttttaaaagaaaaggggggactggaagggctaattcactccca
acgaagacaaaatcgtcgagagatgctgcatataagcagctgctttttgc
ttgtactgggtctctctggttagaccagatctgagcctgggagctctctg
gctaactagggaacccactgcttaagcctcaataaagcttgccttgagtg
cttcaagtagtgtgtgcccgtctgttgtgtgactctggtaactagagatc
cctcagacccttttagtcagtgtggaaaatctctagcagtagtagttcat
gtcatcttattattcagtatttataacttgcaaagaaatgaatatcagag
agtgagaggccttgacattataatagatttagcaggaattgaactaggag
tggagcacacaggcaaagctgcagaagtacttggaagaagccaccagaga
tactcacgattctgcacatacctggctaatcccagatcctaaggattaca
ttaagtttactaacatttatataatgatttatagtttaaagtataaactt
atctaatttactattctgacagatattaattaatcctcaaatatcataag
agatgattactattatccccatttaacacaagaggaaactgagagggaaa
gatgttgaagtaattttcccacaattacagcatccgttagttacgactct
atgatcttctgacacaaattccatttactcctcaccctatgactcagtcg
aatatatcaaagttatggacattatgctaagtaacaaattacccttttat
atagtaaatactgagtagattgagagaagaaattgtttgcaaacctgaat
agcttcaagaagaagagaagtgaggataagaataacagttgtcatttaac
aagttttaacaagtaacttggttagaaagggattcaaatgcataaagcaa
gggataaatttttctggcaacaagactatacaatataaccttaaatatga
cttcaaataattgttggaacttgataaaactaattaaatattattgaaga
ttatcaatattataaatgtaatttacttttaaaaagggaacatagaaatg
tgtatcattagagtagaaaacaatccttattatcacaatttgtcaaaaca
agtttgttattaacacaagtagaatactgcattcaattaagttgactgca
gattttgtgttttgttaaaattagaaagagataacaacaatttgaattat
tgaaagtaacatgtaaatagttctacatacgttcttttgacatcttgttc
aatcattgatcgaagttctttatcttggaagaatttgttccaaagactct
gaaataaggaaaacaatctattatatagtctcacacctttgttttacttt
tagtgatttcaatttaataatgtaaatggttaaaatttattcttctctga
gatcatttcacattgcagatagaaaacctgagactggggtaatttttatt
aaaatctaatttaatctcagaaacacatctttattctaacatcaattttt
ccagtttgatattatcatataaagtcagccttcctcatctgcaggttcca
caacaaaaatccaaccaactgtggatcaaaaatattgggaaaaaattaaa
aatagcaatacaacaataaaaaaatacaaatcagaaaaacagcacagtat
aacaactttatttagcatttacaatctattaggtattataagtaatctag

guide RNA AASV1: SEQ ID NO: 10

ggggcgggcggtgcgatgtcgt

5′ primer: SEQ ID NO: 11

agggccacttctgctaatgg

3′ primer: SEQ ID NO: 12

gataccgtcggcgttggtg

1. A method for the controlled expression of a nucleic acid encoding a CRISPR (Clustered regularly interspaced short palindromic repeats) associated protein (Cas) nuclease in at least one target cell of an individual, limiting the off targets of the Cas nuclease, comprising at least the steps of:
providing to a target cell a nucleic acid encoding a Cas nuclease under the control of a regulatory polynucleotide comprising a minimal promoter and from one to twenty AARE (amino acid response element) nucleic acids; and
inducing the expression of the Cas nuclease by providing a medium deficient in at least one essential amino acid to the target cell.

2. The method according to claim 1, wherein the Cas nuclease is Cas9 nuclease.

3. The method according to claim 1, wherein the amino acid response element (AARE) nucleic acid is selected in a group comprising a nucleic acid of sequence SEQ ID NO: 1, SEQ ID NO: 2, SEQ ID NO: 3, SEQ ID NO: 4 and SEQ ID NO: 5.

4. The method according to claim 1, wherein the regulatory polynucleotide comprises from two to ten AARE nucleic acids.

5. The method according to claim 1, wherein the regulatory polynucleotide comprises from two to six AARE nucleic acids.

6. The method according to claim 1, wherein the regulatory polynucleotide and the nucleic acid encoding a Cas nuclease are included in a nucleic acid vector.

7. The method according to claim 1, wherein the regulatory polynucleotide and the nucleic acid encoding a Cas nuclease are included in a delivery particle.

8. The method according to claim 7, wherein the delivery particle comprises at its surface one or more ligands suitable for binding to a target receptor exposed at a membrane of a targeted cell.

9. A method for editing the genome into at least one target cell of an individual, comprising at least the steps of :
administering to an individual in need thereof a pharmaceutical composition comprising (a) a nucleic acid encoding a Cas nuclease under the control of a regulatory polynucleotide comprising a minimal promoter and from one to twenty AARE (amino acid response element) nucleic acids and (ii) a pharmaceutically acceptable vehicle, and
inducing the expression of the Cas nuclease upon consumption by the individual of a diet deficient in at least one essential amino acid.

10. The method according to claim 9, wherein the Cas nuclease is Cas9 nuclease.

11. The method according to claim 9, wherein the amino acid response element (AARE) nucleic acid is selected in a group comprising a nucleic acid of sequence SEQ ID NO: 1, SEQ ID NO: 2, SEQ ID NO: 3, SEQ ID NO: 4 and SEQ ID NO: 5.

12. The method according to claim 9, wherein the regulatory polynucleotide comprises from two to ten AARE nucleic acids.

13. The method according to claim 9, wherein the regulatory polynucleotide comprises from two to six AARE nucleic acids.

14. The method according to claim 9, wherein the regulatory polynucleotide and the nucleic acid encoding a Cas nuclease are included in a nucleic acid vector.

15. The method according to claim 9, wherein the regulatory polynucleotide and the nucleic acid encoding a Cas nuclease are included in a delivery particle.

16. The method according to claim 15, wherein the delivery particle comprises at its surface one or more ligands suitable for binding to a target receptor exposed at a membrane of the target cell.

17. The method according to claim 9, wherein the pharmaceutical composition further comprises a guide DNA or RNA, which is specific of a target genomic nucleic acid to be edited in the target cell and a donor nucleic acid comprising a nucleic acid intended to replace the target genomic nucleic acid.

18. The method according to claim 17, wherein the target genomic nucleic acid comprises a genetic mutation.

19. A method for preventing and/or treating a disease in an individual in need thereof comprising at least the steps of :
administering to an individual in need thereof a pharmaceutical composition comprising (a) a nucleic acid encoding a Cas nuclease under the control of a regulatory polynucleotide comprising a minimal promoter and from one to twenty AARE (amino acid response element) nucleic acids and (ii) a pharmaceutically acceptable vehicle, and
inducing the expression of the Cas nuclease upon consumption by the individual of a diet deficient in at least one essential amino acid.

20. The method according to claim 19, wherein the Cas nuclease is Cas9 nuclease.

21. The method according to claim 19, wherein the amino acid response element (AARE) nucleic acid is selected in a group comprising a nucleic acid of sequence SEQ ID NO: 1, SEQ ID NO: 2, SEQ ID NO: 3, SEQ ID NO: 4 and SEQ ID NO: 5.

22. The method according to claim 19, wherein the regulatory polynucleotide comprises from two to ten AARE nucleic acids.

23. The method according to claim 19, wherein the regulatory polynucleotide comprises from two to six AARE nucleic acids.

24. The method according to claim 19, wherein the regulatory polynucleotide and the nucleic acid encoding a Cas nuclease are included in a nucleic acid vector.

25. The method according to claim 19, wherein the regulatory polynucleotide and the nucleic acid encoding a Cas nuclease are included in a delivery particle.

26. The method according to claim 25, wherein the delivery particle comprises at its surface one or more ligands suitable for binding to a target receptor exposed at a membrane of a targeted cell.

27. The method according to claim 19, wherein the pharmaceutical composition further comprises a guide DNA or RNA, which is specific of a target genomic nucleic acid to be edited in the target cell and a donor nucleic acid comprising a nucleic acid intended to replace the target genomic nucleic acid.

28. The method according to claim 27, wherein the target genomic nucleic acid comprises a genetic mutation.


SEQUENCE LISTING (12 sequences)

SEQ ID NO: 1 - DNA, 16 bp, synthetic construct
  misc_feature 1..16: note = Synthetic AARE sequence from the TRIB3 gene
  source 1..16: mol_type = other DNA
cggtttgcatcacccg

SEQ ID NO: 2 - DNA, 16 bp, synthetic construct
  misc_feature 1..16: note = Synthetic AARE sequence from the CHOP gene
  source 1..16: mol_type = other DNA
aacattgcatcatccc

SEQ ID NO: 3 - DNA, 16 bp, synthetic construct
  misc_feature 1..16: note = Synthetic AARE sequence from the ASNS gene
  source 1..16: mol_type = other DNA
gaagtttcatcatgcc

SEQ ID NO: 4 - DNA, 16 bp, synthetic construct
  misc_feature 1..16: note = Synthetic AARE sequence from the ATF3 gene
  source 1..16: mol_type = other DNA
agcgttgcatcacccc

SEQ ID NO: 5 - DNA, 16 bp, synthetic construct
  misc_feature 1..16: note = Synthetic AARE sequence from the SNAT2 gene
  source 1..16: mol_type = other DNA
gatattgcatcagttt

SEQ ID NO: 6 - DNA, 94 bp, synthetic construct
  misc_feature 1..94: note = Synthetic Thymidine kinase minimal promoter nucleic acid
  source 1..94: mol_type = other DNA
cgaggtccacttcgcatattaaggtgacgcgtgtggcctcgaacaccgagcgaccctgcagcgacccgcttaacagcgtcaacagcgtgccgca

SEQ ID NO: 7 - DNA, 215 bp, synthetic construct
  misc_feature 1..215: note = Synthetic 2XAARE nucleic acid
  source 1..215: mol_type = other DNA
gattagctccggtttgcatcacccggaccgggggattagctccggtttgcatcacccggaccgggggattagctccggtttgcatcacccggaccgggggccgggcgcgtgctagcgattagctccggtttgcatcacccggaccgggggattagctccggtttgcatcacccggaccgggggattagctccggtttgcatcacccggaccgggg

SEQ ID NO: 8 - DNA, 4212 bp, synthetic construct
  misc_feature 1..4212: note = Synthetic NLS-FLAG CAS9 nucleic acid
  source 1..4212: mol_type = other DNA
atgggacctaagaaaaagaggaaggtgcctaagaaaaagaggaaggtgcctaagaaaaagaggaaggtggcggccgctgactacaaggatgacgacgataaatctagagacaagaaatactctattggactggatatcgggacaaactccgttggctgggccgtcataaccgacgagtataaggtgccaagcaagaaattcaaggtgctgggtaatactgaccgccattcaatcaagaagaacctgatcggagcactcctcttcgactccggtgaaaccgctgaagctactcggctgaagcggaccgcaaggcggagatacacccgccgcaagaatcggatatgttatctgcaagagatctttagcaacgaaatggctaaggtggacgactccttctttcaccgcctggaagagagctttctggtggaggaggataagaaacacgagaggcaccctatattcggaaatatcgtggatgaggtggcttaccatgaaaagtatcctacaatctaccatctgaggaagaagctggtggacagcaccgataaagcagacctgaggctcatctatctggccctggctcatatgataaagtttagaggacactttctgatcgagggcgacctgaatcccgataattccgatgtggataaactcttcattcaactggtgcagacatataaccaactgttcgaggagaatcccataaacgcttctggtgtggatgccaaggctattctgtccgctcggctgtccaagtcacgcagactggagaatctgattgcccaactgccaggagaaaagaagaacggcctgtttgggaacctcatcgccctgagcctgggcctgacacctaacttcaagtccaattttgatctggccgaagatgctaaactccagctctccaaggacacctatgacgatgatctggacaacctgctcgcacagataggcgaccagtacgccgatctctttctggctgctaagaatctctccgacgccattctgctgagcgacatactccgggtcaacactgagatcaccaaagcacctctgagcgcctccatgataaaacgctatgatgaacaccatcaagacctgactctgctcaaagccctcgtgaggcaacagctgccagagaagtacaaagagatattcttcgaccagagcaagaatggatatgccggatacatcgatggcggagcatcacaggaagaattttacaagttcatcaaaccaatcctcgagaagatggacggtactgaagagctgctggtgaagctgaacagggaggacctgctgaggaagcagaggacctttgataatggctccattccacatcagatacacctgggagagctgcatgcaatcctccgcaggcaggaggatttctatcctttcctgaaggataaccgggagaagatagagaagatcctgaccttcaggatcccttattacgtcggccctctggctagaggcaactcccgcttcgcttggatgaccaggaaatctgaggagacaattactccttggaacttcgaagaggtcgtggataagggcgcaagcgcccagtcattcatcgaacggatgaccaatttcgataagaacctgcccaacgagaaggtcctgcccaaacattcactcctgtacgagtatttcaccgtctataacgagctgactaaagtgaagtacgtgaccgagggcatgaggaagcctgccttcctgtccggagagcagaagaaggctatcgttgatctgctcttcaagactaatagaaaggtgacagtgaagcagctcaaggaggattactttaagaagatcgaatgctttgactcagtggaaatctctggcgtggaggaccgctttaatgccagcctgggcacttaccatgatctgctgaagataatcaaagacaaagatttcctcgataatgaggagaacgaggacatcctggaagatatcgtgctgaccctgactctgttcgaggatagagagatgatcgaagagcgcctgaagacctatgcccatctgtttgacgataaagtcatgaaacagctcaagcggcggcgctacactgggtggggtagactctccaggaaactcataaacggcatccgcgacaaacagagcggaaagaccatcctggatttcctgaaatccgacggattcgctaacaggaacttcatgcaactgattcacgatgactctctgacatttaaagaggacatccagaaggcacaggtgagcggtcaaggcgacagcctgcacgagcacatcgccaacctcgctggatcacccgccataaagaagggaatactgcagacagtcaaggtcgtggacgaactcgtcaaagtgatgggtcggcacaagccagagaatatcgttatcgaaatggcaagggagaaccaaaccacccagaagggccagaagaactctcgggaacggatgaaaagaatcgaagagggaattaaggagctgggatctcagatactgaaggagcaccctgtggagaatacacagctccagaacgagaaactctacctgtactacctccagaacgggcgggacatgtacgttgaccaggaactcgacatcaaccggctgtccgattatgacgtggaccatattgttccacagtccttcctcaaagatgactccattgacaacaaggtgctgaccagatccgataagaatcgcggtaagtctgacaatgttccatcagaagaggtggtcaagaagatgaagaattactggcggcagctcctcaacgccaaactgatcacccagcggaagtttgacaatctgactaaggcagaaagaggaggtctgagcgaactcgacaaggccggctttattaagaggcaactggtcgaaacacgccagattaccaaacacgtggcacaaatcctcgactctaggatgaacactaagtacgatgagaacgataagctgatcagggaagtgaaagtgataactctgaagagcaagctggtgtctgacttccggaaggactttcaattctacaaagttcgcgaaataaacaattaccatcatgctcacgatgcctatctcaatgctgtcgttggcaccgccctgatcaagaaataccctaaactggagtctgagttcgtgtacggtgactataaagtctacgatgtgaggaagatgatagcaaagtctgagcaagagattggcaaagccaccgccaagtacttcttctactctaatatcatgaatttctttaagactgagataaccctggctaacggcgaaatccggaagcgcccactgatcgaaacaaacggagaaacaggagaaatcgtgtgggataaaggcagggacttcgcaactgtgcggaaggtgctgtccatgccacaagtcaatatcgtgaagaagaccgaagtgcagaccggcggattctcaaaggagagcatcctgccaaagcggaactctgacaagctgatcgccaggaagaaagattgggacccaaagaagtatggcggtttcgattcccctacagtggcttattccgttctggtcgtggcaaaagtggagaaaggcaagtccaagaaactcaagtctgttaaggagctgctcggaattactattatggagagatccagcttcgagaagaatccaatcgatttcctggaagctaagggctataaagaagtgaagaaagatctcatcatcaaactgcccaagtactctctctttgagctggagaatggtaggaagcggatgctggcctccgccggagagctgcagaaaggaaacgagctggctctgccctccaaatacgtgaacttcctgtatctggcctcccactacgagaaactcaaaggtagccctgaagacaatgagcagaagcaactctttgttgagcaacataaacactacctggacgaaatcattgaacagattagcgagttcagcaagcgggttattctggccgatgcaaacctcgataaagtgctgagcgcatataataagcacagggacaagccaattcgcgaacaagcagagaatattatccacctctttactctgactaatctgggcgctcctgctgccttcaagtatttcgatacaactattgacaggaagcggtacacctctaccaaagaagttctcgatgccaccctgatacaccagtcaattaccggactgtacgagactcgcatcgacctgtctcagctcggcggcgactag

SEQ ID NO: 9 - DNA, 13650 bp, synthetic construct
  misc_feature 1..13650: note = Synthetic Nucleic acid of the pTrip-2XAARE-NLS-FLAG-CAS9 plasmid
  source 1..13650: mol_type = other DNA
ccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcggatctacgcgtcaagtttgtacaaaaaagcaggctccgcggccgcccccttcaccggtaccgattagctccggtttgcatcacccggaccgggggattagctccggtttgcatcacccggaccgggggattagctccggtttgcatcacccggaccgggggccgggcgcgtgctagcgattagctccggtttgcatcacccggaccgggggattagctccggtttgcatcacccggaccgggggattagctccggtttgcatcacccggaccggggactcgaggtccacttcgcatattaaggtgacgcgtgtggcctcgaacaccgagcgaccctgcagcgacccgcttaacagcgtcaacagcgtgccgcaagcttgaattctgatcagcattccggtactgttggtaaagccaccatgggacctaagaaaaagaggaaggtgcctaagaaaaagaggaaggtgcctaagaaaaagaggaaggtggcggccgctgactacaaggatgacgacgataaatctagagacaagaaatactctattggactggatatcgggacaaactccgttggctgggccgtcataaccgacgagtataaggtgccaagcaagaaattcaaggtgctgggtaatactgaccgccattcaatcaagaagaacctgatcggagcactcctcttcgactccggtgaaaccgctgaagctactcggctgaagcggaccgcaaggcggagatacacccgccgcaagaatcggatatgttatctgcaagagatctttagcaacgaaatggctaaggtggacgactccttctttcaccgcctggaagagagctttctggtggaggaggataagaaacacgagaggcaccctatattcggaaatatcgtggatgaggtggcttaccatgaaaagtatcctacaatctaccatctgaggaagaagctggtggacagcaccgataaagcagacctgaggctcatctatctggccctggctcatatgataaagtttagaggacactttctgatcgagggcgacctgaatcccgataattccgatgtggataaactcttcattcaactggtgcagacatataaccaactgttcgaggagaatcccataaacgcttctggtgtggatgccaaggctattctgtccgctcggctgtccaagtcacgcagactggagaatctgattgcccaactgccaggagaaaagaagaacggcctgtttgggaacctcatcgccctgagcctgggcctgacacctaacttcaagtccaattttgatctggccgaagatgctaaactccagctctccaaggacacctatgacgatgatctggacaacctgctcgcacagataggcgaccagtacgccgatctctttctggctgctaagaatctctccgacgccattctgctgagcgacatactccgggtcaacactgagatcaccaaagcacctctgagcgcctccatgataaaacgctatgatgaacaccatcaagacctgactctgctcaaagccctcgtgaggcaacagctgccagagaagtacaaagagatattcttcgaccagagcaagaatggatatgccggatacatcgatggcggagcatcacaggaagaattttacaagttcatcaaaccaatcctcgagaagatggacggtactgaagagctgctggtgaagctgaacagggaggacctgctgaggaagcagaggacctttgataatggctccattccacatcagatacacctgggagagctgcatgcaatcctccgcaggcaggaggatttctatcctttcctgaaggataaccgggagaagatagagaagatcctgaccttcaggatcccttattacgtcggccctctggctagaggcaactcccgcttcgcttggatgaccaggaaatctgaggagacaattactccttggaacttcgaagaggtcgtggataagggcgcaagcgcccagtcattcatcgaacggatgaccaatttcgataagaacctgcccaacgagaaggtcctgcccaaacattcactcctgtacgagtatttcaccgtctataacgagctgactaaagtgaagtacgtgaccgagggcatgaggaagcctgccttcctgtccggagagcagaagaaggctatcgttgatctgctcttcaagactaatagaaaggtgacagtgaagcagctcaaggaggattactttaagaagatcgaatgctttgactcagtggaaatctctggcgtggaggaccgctttaatgccagcctgggcacttaccatgatctgctgaagataatcaaagacaaagatttcctcgataatgaggagaacgaggacatcctggaagatatcgtgctgaccctgactctgttcgaggatagagagatgatcgaagagcgcctgaagacctatgcccatctgtttgacgataaagtcatgaaacagctcaagcggcggcgctacactgggtggggtagactctccaggaaactcataaacggcatccgcgacaaacagagcggaaagaccatcctggatttcctgaaatccgacggattcgctaacaggaacttcatgcaactgattcacgatgactctctgacatttaaagaggacatccagaaggcacaggtgagcggtcaaggcgacagcctgcacgagcacatcgccaacctcgctggatcacccgccataaagaagggaatactgcagacagtcaaggtcgtggacgaactcgtcaaagtgatgggtcggcacaagccagagaatatcgttatcgaaatggcaagggagaaccaaaccacccagaagggccagaagaactctcgggaacggatgaaaagaatcgaagagggaattaaggagctgggatctcagatactgaaggagcaccctgtggagaatacacagctccagaacgagaaactctacctgtactacctccagaacgggcgggacatgtacgttgaccaggaactcgacatcaaccggctgtccgattatgacgtggaccatattgttccacagtccttcctcaaagatgactccattgacaacaaggtgctgaccagatccgataagaatcgcggtaagtctgacaatgttccatcagaagaggtggtcaagaagatgaagaattactggcggcagctcctcaacgccaaactgatcacccagcggaagtttgacaatctgactaaggcagaaagaggaggtctgagcgaactcgacaaggccggctttattaagaggcaactggtcgaaacacgccagattaccaaacacgtggcacaaatcctcgactctaggatgaacactaagtacgatgagaacgataagctgatcagggaagtgaaagtgataactctgaagagcaagctggtgtctgacttccggaaggactttcaattctacaaagttcgcgaaataaacaattaccatcatgctcacgatgcctatctcaatgctgtcgttggcaccgccctgatcaagaaataccctaaactggagtctgagttcgtgtacggtgactataaagtctacgatgtgaggaagatgatagcaaagtctgagcaagagattggcaaagccaccgccaagtacttcttctactctaatatcatgaatttctttaagactgagataaccctggctaacggcgaaatccggaagcgcccactgatcgaaacaaacggagaaacaggagaaatcgtgtgggataaaggcagggacttcgcaactgtgcggaaggtgctgtccatgccacaagtcaatatcgtgaagaagaccgaagtgcagaccggcggattctcaaaggagagcatcctgccaaagcggaactctgacaagctgatcgccaggaagaaagattgggacccaaagaagtatggcggtttcgattcccctacagtggcttattccgttctggtcgtggcaaaagtggagaaaggcaagtccaagaaactcaagtctgttaaggagctgctcggaattactattatggagagatccagcttcgagaagaatccaatcgatttcctggaagctaagggctataaagaagtgaagaaagatctcatcatcaaactgcccaagtactctctctttgagctggagaatggtaggaagcggatgctggcctccgccggagagctgcagaaaggaaacgagctggctctgccctccaaatacgtgaacttcctgtatctggcctcccactacgagaaactcaaaggtagccctgaagacaatgagcagaagcaactctttgttgagcaacataaacactacctggacgaaatcattgaacagattagcgagttcagcaagcgggttattctggccgatgcaaacctcgataaagtgctgagcgcatataataagcacagggacaagccaattcgcgaacaagcagagaatattatccacctctttactctgactaatctgggcgctcctgctgccttcaagtatttcgatacaactattgacaggaagcggtacacctctaccaaagaagttctcgatgccaccctgatacaccagtcaattaccggactgtacgagactcgcatcgacctgtctcagctcggcggcgactagtaaagcggccgggctcgagtctagaaagggtgggcgcgccgacccagctttcttgtacaaagtggctcgacggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctag

SEQ ID NO: 10 - DNA, 22 bp, synthetic construct
  misc_feature 1..22: note = Synthetic guide RNA AASV1
  source 1..22: mol_type = other DNA
ggggcgggcggtgcgatgtcgt

SEQ ID NO: 11 - DNA, 20 bp, synthetic construct
  misc_feature 1..20: note = synthetic 5 prime primer
  source 1..20: mol_type = other DNA
agggccacttctgctaatgg

SEQ ID NO: 12 - DNA, 19 bp, synthetic construct
  misc_feature 1..19: note = Synthetic 3 prime primer
  source 1..19: mol_type = other DNA
gataccgtcggcgttggtg